(19) United States
(12) Patent Application Publication
(10) Pub. No.: US 2011/0190764 A1
(43) Pub. Date: Aug. 4, 2011

(54) SURGICAL INSTRUMENT COMPRISING AN ELECTRODE

(75) Inventors: Gary L. Long, Cincinnati, OH (US); William D. Dannaher, Cincinnati, OH (US); Ragae M. Ghabrial, Loveland, OH (US)

(73) Assignee: Ethicon Endo-Surgery, Inc., Cincinnati, OH (US)

(21) Appl. No.: 12/696,598

(22) Filed: Jan. 29, 2010

(51) Int. Cl. A61B 18/14

(52) U.S. Cl. 606/41

(57) ABSTRACT

A surgical instrument configured to deliver electrical energy to the tissue of a patient, comprising a first electrode comprising a distal portion configured to contact the tissue and a second electrode comprising a distal portion configured to be inserted into the tissue, wherein the distal portion of the second electrode at least partially encompasses the distal portion of the first electrode.

### BACKGROUND

Traditional, or open, surgical techniques may require a surgeon to make large incisions in a patient's body in order to access a tissue treatment region, or surgical site. In some instances, these large incisions may prolong the recovery time of and/or increase the scarring to the patient. As a result, minimally invasive surgical techniques are becoming more preferred among surgeons and patients owing to the reduced size of the incisions required for various procedures. In some circumstances, minimally invasive surgical techniques may reduce the possibility that the patient will suffer undesirable post-surgical conditions, such as scarring and/or infections, for example. Further, such minimally invasive techniques can allow the patient to recover more rapidly as compared to traditional surgical procedures.

Endoscopy is one minimally invasive surgical technique which allows a surgeon to view and evaluate a surgical site by inserting at least one cannula, or trocar, into the patient's body through a natural opening in the body and/or through a relatively small incision. In use, an endoscope can be inserted into, or through, the trocar so that the surgeon can observe the surgical site. In various embodiments, the endoscope may include a flexible or rigid shaft, a camera and/or other suitable optical device, and a handle portion. In at least one embodiment, the optical device can be located on a first, or distal, end of the shaft and the handle portion can be located on a second, or proximal, end of the shaft. In various embodiments, the endoscope may also be configured to assist a surgeon in taking biopsies, retrieving foreign objects, and introducing surgical instruments into the surgical site.

Laparoscopic surgery is another minimally invasive surgical technique where procedures in the abdominal or pelvic cavities can be performed through small incisions in the patient's body. A key element of laparoscopic surgery is the use of a laparoscope which typically includes a telescopic lens system that can be connected to a video camera. In various embodiments, a laparoscope can further include a fiber optic system connected to a halogen or xenon light source, for example, in order to illuminate the surgical site. In various laparoscopic, and/or endoscopic, surgical procedures, a body cavity of a patient, such as the abdominal cavity, for example, can be insufflated with carbon dioxide gas, for example, in order to create a temporary working space for the surgeon. In such procedures, a cavity wall can be elevated above the organs within the cavity by the carbon dioxide gas. Carbon dioxide gas is usually used for insufflation because it can be easily absorbed and removed by the body.

In at least one minimally invasive surgical procedure, an endoscope and/or laparoscope can be inserted through a natural opening of a patient to allow a surgeon to access a surgical site. Such procedures are generally referred to as Nature Orifice Transluminal Endoscopic Surgery or (NOTES)™ and can be utilized to treat tissue while reducing the number of incisions, and external scars, to a patient's body. In various NOTES procedures, for example, an endoscope can include at least one working channel defined therein which can be used to allow the surgeon to insert a surgical instrument therethrough in order to access the surgical site.

The foregoing discussion is intended only to illustrate various aspects of the related art in the field of the invention at the time, and should not be taken as a disavowal of claim scope.

### FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIG. 1 illustrates one embodiment of an electrical ablation system.

FIGS. 2A-D illustrate one embodiment of the electrical ablation system in various phases of deployment.

FIG. 2E illustrates one embodiment of the electrical ablation device comprising multiple needle electrodes.

FIG. 3 illustrates one embodiment of the electrical ablation system shown in FIGS. 1 and 2A-D in use to treat undesirable tissue located on the surface of the liver.

FIG. 4 is an elevational view of a surgical instrument comprising a first electrode and a second electrode, wherein the first electrode encompasses the second electrode.

FIG. 5 is a perspective view of a distal end of the surgical instrument of FIG. 4.

FIG. 6 illustrates the first electrode of the surgical instrument of FIG. 4 positioned against the tissue of a patient.

FIG. 7 illustrates the first electrode and the second electrode of the surgical instrument of FIG. 4 at least partially positioned within the tissue of FIG. 6.

FIG. 8 illustrates a region of tissue that can be treated by the surgical instrument of FIG. 4 as a result of current flowing between the first electrode and the second electrode.

FIG. 9 is an elevational view of an alternative embodiment of a surgical instrument comprising a second electrode that extends distally relative to a second electrode.

FIG. 10 illustrates a region of tissue that can be treated by the surgical instrument of FIG. 9 as a result of current flowing from the second electrode to the first electrode.

FIG. 11 is a perspective view of a distal end of an alternative embodiment of a surgical instrument comprising a first electrode and a second electrode, wherein the second electrode comprises a plurality of annular members connected to one another.

FIG. 12 is an elevational view of the distal end of the surgical instrument of FIG. 11.

FIG. 13 is a perspective view of a distal end of an alternative embodiment of a surgical instrument comprising a first electrode and a second electrode, wherein a first fluid conducting passageway is present in the first electrode and wherein a second fluid conducting passageway is present in the second electrode.

FIG. 14 is an elevational view of the surgical instrument of FIG. 13.

FIG. 15 is an elevational view of a distal end of an alternative embodiment of a surgical instrument comprising an electrode having a plurality of helical windings, wherein certain helical windings have a first pitch angle and other helical windings have a different pitch angle.

FIG. 16 is an elevational view of a distal end of an alternative embodiment of a surgical instrument comprising an electrode having a plurality of helical windings, wherein the distal end of the electrode extends downwardly at a different angle than the pitch angle of the windings.

FIG. 17 is an elevational view of a surgical instrument comprising a motor configured to rotate an electrode.

Corresponding reference characters indicate corresponding parts throughout the several views. The exemplifications set out herein illustrate various embodiments of the invention, in one form, and such exemplifications are not to be construed as limiting the scope of the invention in any manner.

### DESCRIPTION

Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Various embodiments are directed to apparatuses, systems, and methods for the electrical ablation treatment of undesirable tissue such as diseased tissue, cancer, malignant and benign tumors, masses, lesions, and other abnormal tissue growths. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without the specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Electrical ablation devices in accordance with the described embodiments may comprise one or more electrodes configured to be positioned into or proximal to undesirable tissue in a tissue treatment region (e.g., target site, worksite) where there is evidence of abnormal tissue growth, for example. In general, the electrodes comprise an electrically conductive portion (e.g., medical grade stainless steel) and are configured to electrically couple to an energy source. Once the electrodes are positioned into or proximal to the undesirable tissue, an energizing potential is applied to the electrodes to create an electric field to which the undesirable tissue is exposed. The energizing potential (and the resulting electric field) may be characterized by multiple parameters such as frequency, amplitude, pulse width (duration of a pulse or pulse length), and/or polarity. Depending on the diagnostic or therapeutic treatment to be rendered, a particular electrode may be configured either as an anode (+) or a cathode (−) or may comprise a plurality of electrodes with at least one configured as an anode and at least one other configured as a cathode. Regardless of the initial polar configuration, the polarity of the electrodes may be reversed by reversing the polarity of the output of the energy source.

In various embodiments, a suitable energy source may comprise an electrical waveform generator, which may be configured to create an electric field that is suitable to create irreversible electroporation in undesirable tissue at various electric filed amplitudes and durations. The energy source may be configured to deliver irreversible electroporation pulses in the form of direct-current (DC) and/or alternating-current (AC) voltage potentials (e.g., time-varying voltage potentials) to the electrodes. The irreversible electroporation pulses may be characterized by various parameters such as frequency, amplitude, pulse length, and/or polarity. The undesirable tissue may be ablated by exposure to the electric potential difference across the electrodes.

In one embodiment, the energy source may comprise a wireless transmitter to deliver energy to the electrodes using wireless energy transfer techniques via one or more remotely positioned antennas. Those skilled in the art will appreciate that wireless energy transfer or wireless power transmission is the process of transmitting electrical energy from an energy source to an electrical load without interconnecting wires. An electrical transformer is the simplest instance of wireless energy transfer. The primary and secondary circuits of a transformer are not directly connected and the transfer of energy takes place by electromagnetic coupling through a process known as mutual induction. Power also may be transferred wirelessly using RF energy. Wireless power transfer technology using RF energy is produced by Powercast, Inc. and can achieve an output of 6 volts for a little over one meter. Other low-power wireless power technology has been proposed such as described in U.S. Pat. No. 6,967,462, the entire disclosure of which is incorporated by reference herein.

The apparatuses, systems, and methods in accordance with certain described embodiments may be configured for minimally invasive ablation treatment of undesirable tissue through the use of irreversible electroporation to be able to ablate undesirable tissue in a controlled and focused manner without inducing thermally damaging effects to the surrounding healthy tissue. The apparatuses, systems, and methods in accordance with the described embodiments may be configured to ablate undesirable tissue through the use of electroporation or electropermeabilization. More specifically, in various embodiments, the apparatuses, systems, and methods in accordance with the described embodiments may be configured to ablate undesirable tissue through the use of irreversible electroporation. Electroporation increases the permeabilization of a cell membrane by exposing the cell to electric pulses. The external electric field (electric potential/per unit length) to which the cell membrane is exposed to significantly increases the electrical conductivity and permeability of the plasma in the cell membrane. The primary parameter affecting the transmembrane potential is the potential difference across the cell membrane. Irreversible electroporation is the application of an electric field of a specific magnitude and duration to a cell membrane such that the permeabilization of the cell membrane cannot be reversed, leading to cell death without inducing a significant amount of heat in the cell membrane. The destabilizing potential forms pores in the cell membrane when the potential across the cell membrane exceeds its dielectric strength causing the cell to die under a process known as apoptosis and/or necrosis. The application of irreversible electroporation pulses to cells is an effective way for ablating large volumes of undesirable tissue without deleterious thermal effects to the surrounding healthy tissue associated with thermal-inducing ablation treatments. This is because irreversible electroporation destroys cells without heat and thus does not destroy the cellular support structure or regional vasculature. A destabilizing irreversible electroporation pulse, suitable to cause cell death without inducing a significant amount of thermal damage to the surrounding healthy tissue, may have amplitude in the range of about several hundred to about several thousand volts and is generally applied across biological membranes over a distance of about several millimeters, for example, for a relatively long duration. Thus, the undesirable tissue may be ablated in-vivo through the delivery of destabilizing electric fields by quickly creating cell necrosis.

The apparatuses, systems, and methods for electrical ablation therapy in accordance with the described embodiments may be adapted for use in minimally invasive surgical procedures to access the tissue treatment region in various anatomic locations such as the brain, lungs, breast, liver, gall bladder, pancreas, prostate gland, and various internal body lumen defined by the esophagus, stomach, intestine, colon, arteries, veins, anus, vagina, cervix, fallopian tubes, and the peritoneal cavity, for example, without limitation. Minimally invasive electrical ablation devices may be introduced to the tissue treatment region using a trocar inserted though a small opening formed in the patient's body or through a natural body orifice such as the mouth, anus, or vagina using translumenal access techniques known as Natural Orifice Translumenal Endoscopic Surgery (NOTES)™. Once the electrical ablation devices (e.g., electrodes) are located into or proximal to the undesirable tissue in the treatment region, electric field potentials can be applied to the undesirable tissue by the energy source. The electrical ablation devices comprise portions that may be inserted into the tissue treatment region percutaneously (e.g., where access to inner organs or other tissue is done via needle-puncture of the skin). Other portions of the electrical ablation devices may be introduced into the tissue treatment region endoscopically (e.g., laparoscopically and/or thoracoscopically) through trocars or working channels of the endoscope, through small incisions, or transcutaneously (e.g., where electric pulses are delivered to the tissue treatment region through the skin).

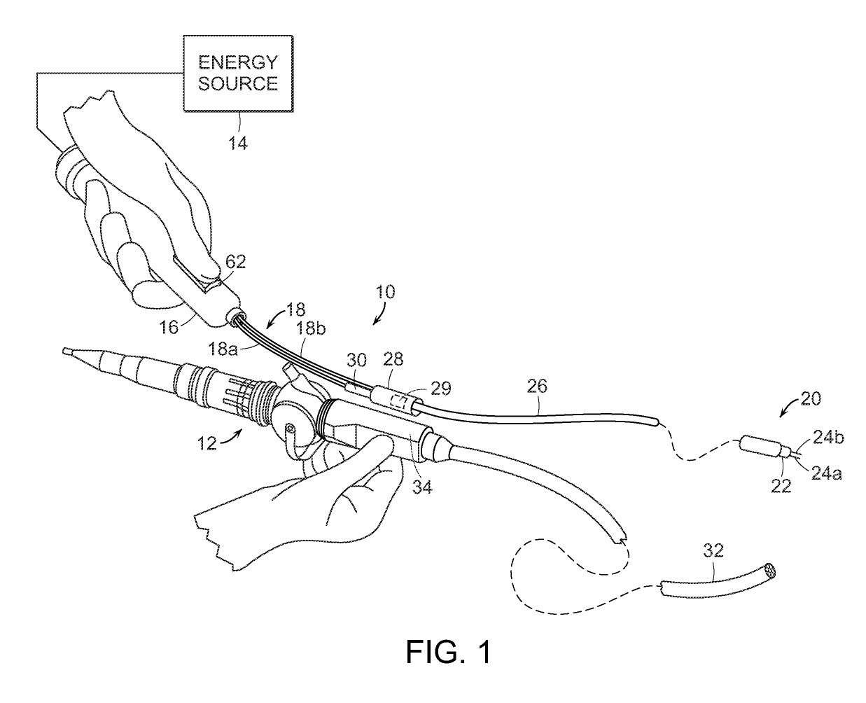

FIG. 1 illustrates one embodiment of an electrical ablation system 10. The electrical ablation system 10 may be employed to ablate undesirable tissue such as diseased tissues, cancers, tumors, masses, lesions, abnormal tissue growths inside a patient using electrical energy. The electrical ablation system 10 may be used in conjunction with endoscopic, laparoscopic, thoracoscopic, open surgical procedures via small incisions or keyholes, percutaneous techniques, transcutaneous techniques, and/or external non-invasive techniques, or any combinations thereof without limitation. The electrical ablation system 10 may be configured to be positioned within a natural body orifice of the patient such as the mouth, anus, or vagina and advanced through internal body lumen or cavities such as the esophagus, colon, cervix, urethra, for example, to reach the tissue treatment region. The electrical ablation system 10 also may be configured to be positioned and passed through a small incision or keyhole formed through the skin or abdominal wall of the patient to reach the tissue treatment region using a trocar. The tissue treatment region may be located in the brain, lungs, breast, liver, gall bladder, pancreas, prostate gland, various internal body lumen defined by the esophagus, stomach, intestine, colon, arteries, veins, anus, vagina, cervix, fallopian tubes, and the peritoneal cavity, for example, without limitation. The electrical ablation system 10 can be configured to treat a number of lesions and ostepathologies comprising metastatic lesions, tumors, fractures, infected sites, inflamed sites. Once positioned into or proximate the tissue treatment region, the electrical ablation system 10 can be actuated (e.g., energized) to ablate the undesirable tissue. In one embodiment, the electrical ablation system 10 may be configured to treat diseased tissue in the gastrointestinal (GI) tract, esophagus, lung, or stomach that may be accessed orally. In another embodiment, the electrical ablation system 10 may be adapted to treat undesirable tissue in the liver or other organs that may be accessible using translumenal access techniques such as, without limitation, NOTES™ techniques, where the electrical ablation devices may be initially introduced through a natural orifice such as the mouth, anus, or vagina and then advanced to the tissue treatment site by puncturing the walls of internal body lumen such as the stomach, intestines, colon, cervix. In various embodiments, the electrical ablation system 10 may be adapted to treat undesirable tissue in the brain, liver, breast, gall bladder, pancreas, or prostate gland, using one or more electrodes positioned percutaneously, transcutaneously, translumenally, minimally invasively, and/or through open surgical techniques, or any combination thereof.

In one embodiment, the electrical ablation system 10 may be employed in conjunction with a flexible endoscope 12, as well as a rigid endoscope, laparoscope, or thoracoscope, such as the GIF-100 model available from Olympus Corporation. In one embodiment, the endoscope 12 may be introduced to the tissue treatment region trans-anally through the colon, trans-orally through the esophagus and stomach, trans-vaginally through the cervix, transcutaneously, or via an external incision or keyhole formed in the abdomen in conjunction with a trocar. The electrical ablation system 10 may be inserted and guided into or proximate the tissue treatment region using the endoscope 12.

In the embodiment illustrated in FIG. 1, the endoscope 12 comprises an endoscope handle 34 and an elongate relatively flexible shaft 32. The distal end of the flexible shaft 32 may comprise a light source and a viewing port. Optionally, the flexible shaft 32 may define one or more working channels for receiving various instruments, such as electrical ablation devices, for example, therethrough. Images within the field of view of the viewing port are received by an optical device, such as a camera comprising a charge coupled device (CCD) usually located within the endoscope 12, and are transmitted to a display monitor (not shown) outside the patient.

In one embodiment, the electrical ablation system 10 may comprise an electrical ablation device 20, a plurality of electrical conductors 18, a handpiece 16 comprising an activation switch 62, and an energy source 14, such as an electrical waveform generator, electrically coupled to the activation switch 62 and the electrical ablation device 20. The electrical ablation device 20 comprises a relatively flexible member or shaft 22 that may be introduced to the tissue treatment region using a variety of known techniques such as an open incision and a trocar, through one of more of the working channels of the endoscope 12, percutaneously, or transcutaneously, for example.

In one embodiment, one or more electrodes (e.g., needle electrodes, balloon electrodes), such as first and second electrodes 24a,b, extend out from the distal end of the electrical ablation device 20. In one embodiment, the first electrode 24a may be configured as the positive electrode and the second electrode 24b may be configured as the negative electrode. The first electrode 24a is electrically connected to a first electrical conductor 18a, or similar electrically conductive lead or wire, which is coupled to the positive terminal of the energy source 14 through the activation switch 62. The second electrode 24b is electrically connected to a second electrical conductor 18b, or similar electrically conductive lead or wire, which is coupled to the negative terminal of the energy source 14 through the activation switch 62. The electrical conductors 18a,b are electrically insulated from each other and surrounding structures, except for the electrical connections to the respective electrodes 24a,b. In various embodiments, the electrical ablation device 20 may be configured to be introduced into or proximate the tissue treatment region using the endoscope 12 (laparoscope or thoracoscope), open surgical procedures, or external and non-invasive medical procedures. The electrodes 24a,b may be referred to herein as endoscopic or laparoscopic electrodes, although variations thereof may be inserted transcutaneously or percutaneously. As previously discussed, either one or both electrodes 24a,b may be adapted and configured to slideably move in and out of a cannula, lumen, or channel defined within the flexible shaft 22.

Once the electrodes 24a,b are positioned at the desired location into or proximate the tissue treatment region, the electrodes 24a,b may be connected to or disconnected from the energy source 14 by actuating or de-actuating the switch 62 on the handpiece 16. The switch 62 may be operated manually or may be mounted on a foot switch (not shown), for example. The electrodes 24a,b deliver electric field pulses to the undesirable tissue. The electric field pulses may be characterized based on various parameters such as pulse shape, amplitude, frequency, and duration. The electric field pulses may be sufficient to induce irreversible electroporation in the undesirable tissue. The induced potential depends on a variety of conditions such as tissue type, cell size, and electrical pulse parameters. The primary electrical pulse parameter affecting the transmembrane potential for a specific tissue type is the amplitude of the electric field and pulse length that the tissue is exposed to.

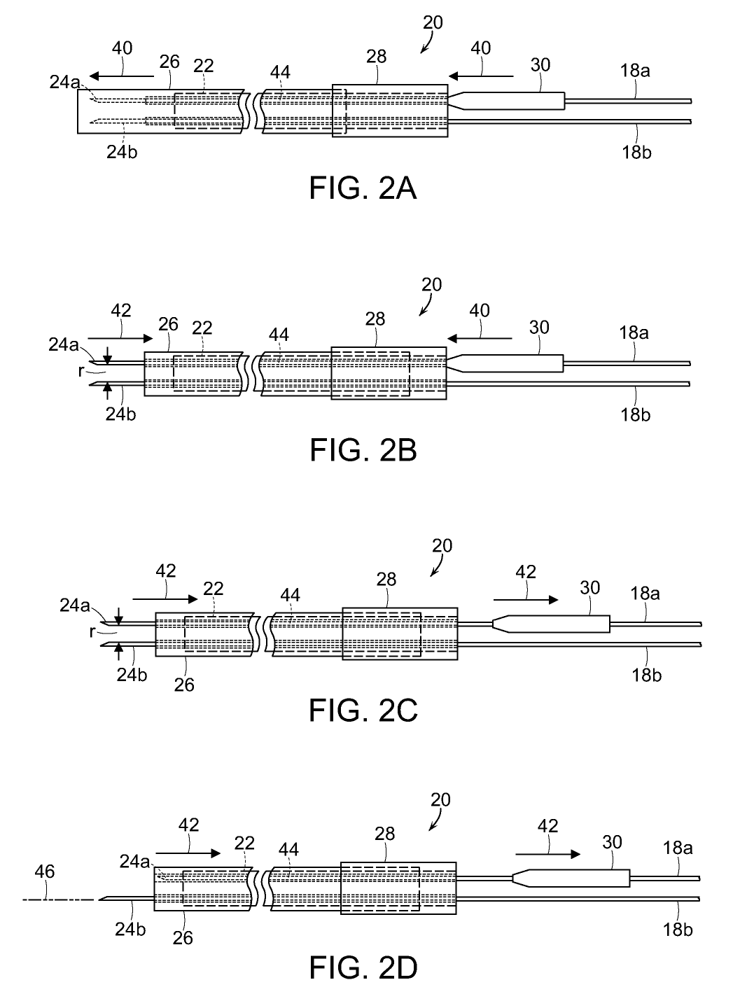

In one embodiment, a protective sleeve or sheath 26 may be slidably disposed over the flexible shaft 22 and within a handle 28. In another embodiment, the sheath 26 may be slidably disposed within the flexible shaft 22 and the handle 28, without limitation. The sheath 26 is slideable and may be located over the electrodes 24a,b to protect the trocar and prevent accidental piercing when the electrical ablation device 20 is advanced therethrough. Either one or both of the electrodes 24a,b of the electrical ablation device 20 may be adapted and configured to slideably move in and out of a cannula, lumen, or channel formed within the flexible shaft 22. The second electrode 24b may be fixed in place. The second electrode 24b may provide a pivot about which the first electrode 24a can be moved in an arc to other points in the tissue treatment region to treat larger portions of the diseased tissue that cannot be treated by fixing the electrodes 24a,b in one location. In one embodiment, either one or both of the electrodes 24a,b may be adapted and configured to slideably move in and out of a working channel formed within a flexible shaft 32 of the flexible endoscope 12 or may be located independently of the flexible endoscope 12. Various features of the first and second electrodes 24a,b are described in more detail in FIGS. 2A-D.

In one embodiment, the first and second electrical conductors 18a,b may be provided through the handle 28. In the illustrated embodiment, the first electrode 24a can be slideably moved in and out of the distal end of the flexible shaft 22 using a slide member 30 to retract and/or advance the first electrode 24a. In various embodiments either or both electrodes 24a,b may be coupled to the slide member 30, or additional slide members, to advance and retract the electrodes 24a,b, e.g., position the electrodes 24a,b. In the illustrated embodiment, the first electrical conductor 18a coupled to the first electrode 24a is coupled to the slide member 30. In this manner, the first electrode 24a, which is slidably movable within the cannula, lumen, or channel defined by the flexible shaft 22, can advanced and retracted with the slide member 30.

In various other embodiments, transducers or sensors 29 may be located in the handle 28 of the electrical ablation device 20 to sense the force with which the electrodes 24a,b penetrate the tissue in the tissue treatment zone. This feedback information may be useful to determine whether either one or both of the electrodes 24a,b have been properly inserted in the tissue treatment region. As is particularly well known, cancerous tumor tissue tends to be denser than healthy tissue and thus greater force is required to insert the electrodes 24a,b therein. The transducers or sensors 29 can provide feedback to the operator, surgeon, or clinician to physically sense when the electrodes 24a,b are placed within the cancerous tumor. The feedback information provided by the transducers or sensors 29 may be processed and displayed by circuits located either internally or externally to the energy source 14. The sensor 29 readings may be employed to determine whether the electrodes 24a,b have been properly located within the cancerous tumor thereby assuring that a suitable margin of error has been achieved in locating the electrodes 24a,b.

In one embodiment, the input to the energy source 14 may be connected to a commercial power supply by way of a plug (not shown). The output of the energy source 14 is coupled to the electrodes 24a,b, which may be energized using the activation switch 62 on the handpiece 16, or in one embodiment, an activation switch mounted on a foot activated pedal (not shown). The energy source 14 may be configured to produce electrical energy suitable for electrical ablation, as described in more detail below.

In one embodiment, the electrodes 24a,b are adapted and configured to electrically couple to the energy source 14 (e.g., generator, waveform generator). Once electrical energy is coupled to the electrodes 24a,b, an electric field is formed at a distal end of the electrodes 24a,b. The energy source 14 may be configured to generate electric pulses at a predetermined frequency, amplitude, pulse length, and/or polarity that are suitable to induce irreversible electroporation to ablate substantial volumes of undesirable tissue in the treatment region. For example, the energy source 14 may be configured to deliver DC electric pulses having a predetermined frequency, amplitude, pulse length, and/or polarity suitable to induce irreversible electroporation to ablate substantial volumes of undesirable tissue in the treatment region. The DC pulses may be positive or negative relative to a particular reference polarity. The polarity of the DC pulses may be reversed or inverted from positive-to-negative or negative-to-positive a predetermined number of times to induce irreversible electroporation to ablate substantial volumes of undesirable tissue in the treatment region.

In one embodiment, a timing circuit may be coupled to the output of the energy source 14 to generate electric pulses. The timing circuit may comprise one or more suitable switching elements to produce the electric pulses. For example, the energy source 14 may produce a series of n electric pulses (where n is any positive integer) of sufficient amplitude and duration to induce irreversible electroporation suitable for tissue ablation when the n electric pulses are applied to the electrodes 24a,b. In one embodiment, the electric pulses may have a fixed or variable pulse length, amplitude, and/or frequency.

The electrical ablation device 20 may be operated either in bipolar or monopolar mode. In bipolar mode, the first electrode 24a is electrically connected to a first polarity and the second electrode 24b is electrically connected to the opposite polarity. For example, in monopolar mode, the first electrode 24a is coupled to a prescribed voltage and the second electrode 24b is set to ground. In the illustrated embodiment, the energy source 14 may be configured to operate in either the bipolar or monopolar modes with the electrical ablation system 10. In bipolar mode, the first electrode 24a is electrically connected to a prescribed voltage of one polarity and the second electrode 24b is electrically connected to a prescribed voltage of the opposite polarity. When more than two electrodes are used, the polarity of the electrodes may be alternated so that any two adjacent electrodes may have either the same or opposite polarities, for example.

In monopolar mode, it is not necessary that the patient be grounded with a grounding pad. Since a monopolar energy source 14 is typically constructed to operate upon sensing a ground pad connection to the patient, the negative electrode of the energy source 14 may be coupled to an impedance simulation circuit. In this manner, the impedance circuit simulates a connection to the ground pad and thus is able to activate the energy source 14. It will be appreciated that in monopolar mode, the impedance circuit can be electrically connected in series with either one of the electrodes 24a,b that would otherwise be attached to a grounding pad.

In one embodiment, the energy source 14 may be configured to produce RF waveforms at predetermined frequencies, amplitudes, pulse widths or durations, and/or polarities suitable for electrical ablation of cells in the tissue treatment region. One example of a suitable RF energy source is a commercially available conventional, bipolar/monopolar electrosurgical RF generator such as Model Number ICC 350, available from Erbe, GmbH.

In one embodiment, the energy source 14 may be configured to produce destabilizing electrical potentials (e.g., fields) suitable to induce irreversible electroporation. The destabilizing electrical potentials may be in the form of bipolar/monopolar DC electric pulses suitable for inducing irreversible electroporation to ablate tissue undesirable tissue with the electrical ablation device 20. A commercially available energy source suitable for generating irreversible electroporation electric filed pulses in bipolar or monopolar mode is a pulsed DC generator such as Model Number ECM 830, available from BTX Molecular Delivery Systems Boston, Mass. In bipolar mode, the first electrode 24a may be electrically coupled to a first polarity and the second electrode 24b may be electrically coupled to a second (e.g., opposite) polarity of the energy source 14. Bipolar/monopolar DC electric pulses may be produced at a variety of frequencies, amplitudes, pulse lengths, and/or polarities. Unlike RF ablation systems, however, which require high power and energy levels delivered into the tissue to heat and thermally destroy the tissue, irreversible electroporation requires very little energy input into the tissue to kill the undesirable tissue without the detrimental thermal effects because with irreversible electroporation the cells are destroyed by electric field potentials rather than heat.

In one embodiment, the energy source 14 may be coupled to the first and second electrodes 24a,b by either a wired or a wireless connection. In a wired connection, the energy source 14 is coupled to the electrodes 24a,b by way of the electrical conductors 18a,b, as shown. In a wireless connection, the electrical conductors 18a,b may be replaced with a first antenna (not shown) coupled the energy source 14 and a second antenna (not shown) coupled to the electrodes 24a,b, wherein the second antenna is remotely located from the first antenna.

In one embodiment, the energy source may comprise a wireless transmitter to deliver energy to the electrodes using wireless energy transfer techniques via one or more remotely positioned antennas. As previously discussed, wireless energy transfer or wireless power transmission is the process of transmitting electrical energy from the energy source 14 to an electrical load, e.g., the abnormal cells in the tissue treatment region, without using the interconnecting electrical conductors 18a,b. An electrical transformer is the simplest instance of wireless energy transfer. The primary and secondary circuits of a transformer are not directly connected. The transfer of energy takes place by electromagnetic coupling through a process known as mutual induction. Wireless power transfer technology using RF energy is produced by Powercast, Inc. The Powercast system can achieve a maximum output of 6 volts for a little over one meter. Other low-power wireless power technology has been proposed such as described in U.S. Pat. No. 6,967,462.

In one embodiment, the energy source 14 may be configured to produce DC electric pulses at frequencies in the range of about 1 Hz to about 10000 Hz, amplitudes in the range of about ±100 to about ±3000 VDC, and pulse lengths (e.g., pulse width, pulse duration) in the range of about 1 μs to about 100 ms. The polarity of the electric potentials coupled to the electrodes 24a,b may be reversed during the electrical ablation therapy. For example, initially, the DC electric pulses may have a positive polarity and an amplitude in the range of about +100 to about +3000 VDC. Subsequently, the polarity of the DC electric pulses may be reversed such that the amplitude is in the range of about −100 to about −3000 VDC. In one embodiment, the undesirable cells in the tissue treatment region may be electrically ablated with DC pulses suitable to induce irreversible electroporation at frequencies of about 10 Hz to about 100 Hz, amplitudes in the range of about +700 to about +1500 VDC, and pulse lengths of about 10 ps to about 50 μs. In another embodiment, the abnormal cells in the tissue treatment region may be electrically ablated with an electrical waveform having an amplitude of about +500 VDC and pulse duration of about 20 ms delivered at a pulse period T or repetition rate, frequency f=1/T, of about 10 Hz. It has been determined that an electric field strength of 1,000 V/cm is suitable for destroying living tissue by inducing irreversible electroporation.

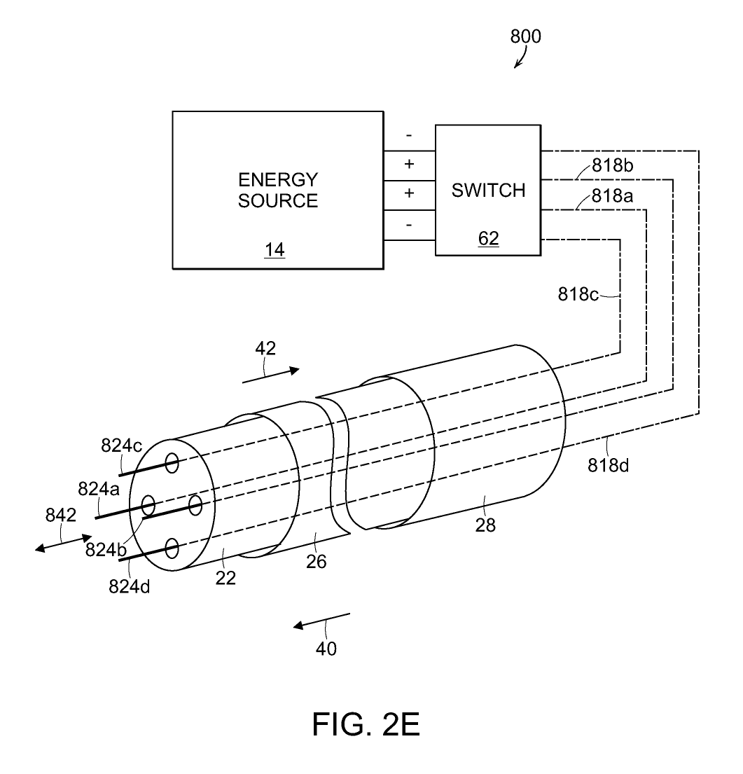

FIGS. 2A-D illustrate one embodiment of the electrical ablation device 20 in various phases of deployment. In the embodiment illustrated in FIGS. 2A-D, the sheath 26 is disposed over the flexible shaft 22, however, those skilled in the art will appreciate that the sheath 26 may be disposed within the flexible shaft 22. The electrical ablation device 20 may be used in conjunction with the electrical ablation system 10 shown in FIG. 1. It will be appreciated that other devices and electrode configurations may be employed without limitation. FIG. 2A illustrates an initial phase of deployment wherein the sheath 26 is extended in the direction indicated by arrow 40 to cover the electrodes 24a,b. The electrodes 24a,b may have dimensions of about 0.5 mm, about 1 mm, or about 1.5 mm in diameter. It will be appreciated that the dimensions of the electrodes 24a,b may be anywhere from about 0.5 mm to about 1.5 mm in diameter. The electrical ablation device 20 may be introduced into the tissue treatment region through a trocar, as illustrated in FIG. 3, for example. FIG. 2B illustrates another phase of deployment wherein the sheath 26 is retracted within the handle 28 in the direction indicated by arrow 42. In this phase of deployment, the first and second electrodes 24a,b extend through the distal end of the flexible shaft 22 and are ready to be inserted into or proximate the tissue treatment region. The first electrode 24a may be retracted in direction 42 through a lumen 44 formed in the flexible shaft 22 by holding the handle 28 and pulling on the slide member 30. FIG. 2C illustrates a transition phase wherein the first electrode 24a is the process of being retracted in direction 42 by pulling on the slide member 30 handle, for example, in the same direction. FIG. 2D illustrates another phase of deployment wherein the first electrode 24a is in a fully retracted position. In this phase of deployment the electrical ablation device 20 can be pivotally rotated about an axis 46 defined by the second electrode 24b. The electrodes 24a,b are spaced apart by a distance “r.” The distance “r” between the electrodes 24a,b may be 5.0 mm, about 7.5 mm, or about 10 mm. It will be appreciated that the distance “r” between the electrodes 24a,b may be anywhere from about 5.0 mm to about 10.0 mm. Thus, the electrical ablation device 20 may be rotated in an arc about the pivot formed by the second electrode 24b, the first electrode 24a may be placed in a new location in the tissue treatment region within the radius “r.” Retracting the first electrode 24a and pivoting about the second electrode 24b enables the surgeon or clinician to target and treat a larger tissue treatment region essentially comprising a circular region having a radius “r,” which is the distance between the electrodes 24a,b. Thus, the electrodes 24a,b may be located in a plurality of positions in and around the tissue treatment region in order to treat much larger regions of tissue. Increasing the electrode 24a,b diameter and spacing the electrodes 24a,b further apart enables the generation of an electric field over a much larger tissue regions and thus the ablation of larger volumes of undesirable tissue. In this manner, the operator can treat a larger tissue treatment region comprising cancerous lesions, polyps, or tumors, for example.

Although the electrical ablation electrodes according to the described embodiments have been described in terms of the particular needle type electrodes 24a,b as shown and described in FIGS. 1 and 2A-D, those skilled in the art will appreciate that other configurations of electrical ablation electrodes may be employed for the ablation of undesirable tissue, without limitation. In one embodiment, the electrical ablation device 20 may comprise two or more fixed electrodes that are non-retractable. In another embodiment, the electrical ablation device 20 may comprise two or more retractable electrodes, one embodiment of which is described below with reference to FIG. 2E. In another embodiment, the electrical ablation device 20 may comprise at least one slidable electrode disposed within at least one working channel of the flexible shaft 32 of the endoscope 12. In another embodiment, the electrical ablation device 20 may comprise at least one electrode may be configured to be inserted into the tissue treatment region transcutaneously or percutaneously. Still in various other embodiments, the electrical ablation device 20 may comprise at least one electrode configured to be introduced to the tissue treatment region transcutaneously or percutaneously and at least one other electrode may be configured to be introduced to the tissue treatment region through at least one working channel of the flexible shaft 32 of the endoscope 12. The embodiments, however, are not limited in this context.

FIG. 2E illustrates one embodiment of an electrical ablation device 100 comprising multiple needle electrodes 124m, where m is any positive integer. In the illustrated embodiment, the electrical ablation device 100 comprises four electrodes 124a, 124b, 124c, 124d. It will be appreciated that in one embodiment, the electrical ablation device 800 also may comprise three needle electrodes 124a, 124b, 124c, without limitation. The electrical ablation device 100 may be used in conjunction with the electrical ablation system 10 shown in FIG. 1. It will be appreciated that other devices and electrode configurations may be employed without limitation. The electrodes 124a-m each may have dimensions of about 0.5 mm, about 1 mm, or about 1.5 mm in diameter. It will be appreciated that the dimensions of each of the electrodes 124a-m may be anywhere from about 0.5 mm to about 1.5 mm in diameter. The electrical ablation device 100 may be introduced into the tissue treatment region through a trocar, as subsequently described and illustrated with reference to FIG. 3, for example.

The electrical ablation device 100 comprises essentially the same components as the electrical ablation device 20 described with reference to FIGS. 2A-D. The electrical ablation device 100 comprises the relatively flexible member or shaft 22, the protective sheath 26, and one or more handles 28 to operate either the sheath 26, the electrodes 124a,b,c,d, or both. The electrodes 124a,b,c,d may be individually or simultaneously deployable and/or retractable in the direction indicated by arrow 142. The electrodes 124a,b,c,d extend out from the distal end of the electrical ablation device 100. In one embodiment, the first and second electrodes 124a, 124b may be configured as the positive electrode coupled to the anode of the energy source 14 via corresponding first and second electrical conductors 118a, 118b, and the third and fourth 124c, 124d may be configured as the negative electrode coupled to the cathode of the energy source 14 via corresponding third and fourth electrical conductors 118c, 118d, or similar electrically conductive leads or wires, through the activation switch 62. Once the electrodes 124a,b,c,d are positioned at the desired location into or proximate the tissue treatment region, the electrodes 124a,b,c,d may be connected/disconnected from the energy source 14 by actuating/de-actuating the switch 62.

As previously discussed with reference to FIGS. 2A-D, as shown in FIG. 2E in one embodiment, the protective sleeve or sheath 26 may be slidably disposed over the flexible shaft 22 and within the handle 28. In an initial phase of deployment, the sheath 26 is extended in direction 40 to cover the electrodes 124a,b,c,d to protect the trocar and prevent accidental piercing when the electrical ablation device 100 is advanced therethrough. Once the electrodes 124a,b,c,d are located into or proximate the tissue treatment region, the sheath 26 is retracted in direction 42 to expose the electrodes 124a,b,c,d. One or more of the electrodes 124a,b,c,d of the electrical ablation device 100 may be adapted and configured to slideably move in and out of a cannula, lumen, or channel formed within the flexible shaft 22. In one embodiment all of the electrodes 124a,b,c,d are configured to slideably move in and out channels formed within lumens formed within the flexible shaft 22, referred to for example as the lumen 44 in FIGS. 2A-D, to advance and retract the electrodes 124a,b,c,d as may be desired by the operator. Nevertheless, in other embodiments, it may be desired to fix all or certain ones of the one or more electrodes 124a,b,c,d in place.

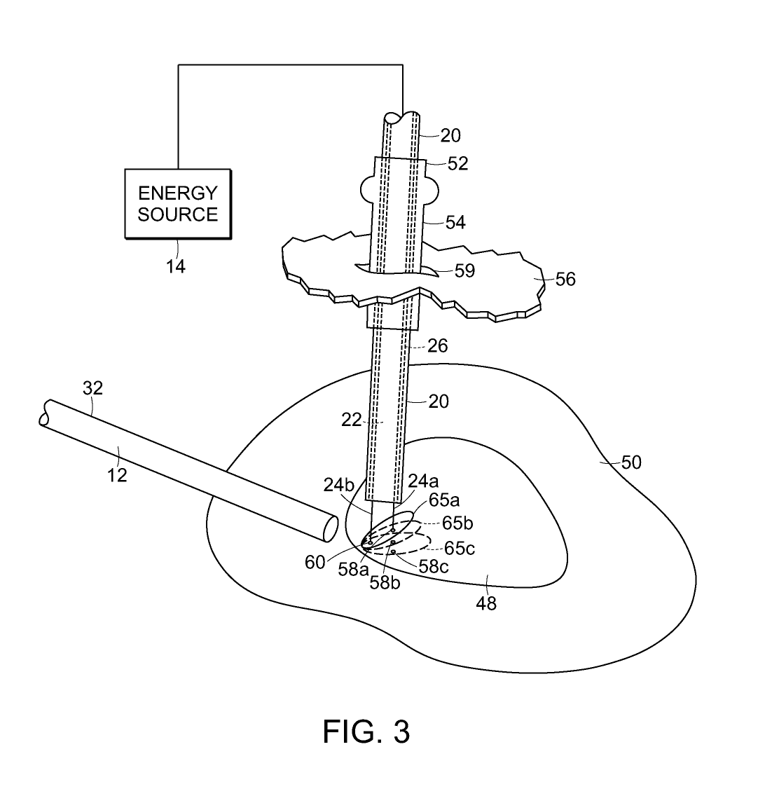

The various embodiments of electrodes described in the present specification, e.g., the electrodes 24a,b, or 124a-m, may be configured for use with an electrical ablation device (not shown) comprising an elongated flexible shaft to house the needle electrodes 24a,b, or 124a-m, for example. The needle electrodes 24a,b, or 124a-m, are free to extend past a distal end of the electrical ablation device. The flexible shaft comprises multiple lumen formed therein to slidably receive the needle electrodes 24a,b, or 124a-m. A flexible sheath extends longitudinally from a handle portion to the distal end. The handle portion comprises multiple slide members received in respective slots defining respective walls. The slide members are coupled to the respective needle electrodes 24a,b, or 124a-m. The slide members are movable to advance and retract the electrode 24a,b, or 124a-m. The needle electrodes 24a,b, or 124a-m, may be independently movable by way of the respective slide members. The needle electrodes 24a,b, or 124a-m, may be deployed independently or simultaneously. An electrical ablation device (not shown) comprising an elongated flexible shaft to house multiple needle electrodes and a suitable handle is described with reference to FIGS. 4-10 in commonly owned U.S. patent application Ser. No. 11/897,676 titled “ELECTRICAL ABLATION SURGICAL INSTRUMENTS,” filed Aug. 31, 2007, the entire'disclosure of which is incorporated herein by reference in its entirety.

It will be appreciated that the electrical ablation devices 20, 100 described with referenced to FIGS. 2A-E, may be introduced inside a patient endoscopically, transcutaneously, percutaneously, through an open incision, through a trocar (as shown in FIG. 3), through a natural orifice, or any combination thereof. In one embodiment, the outside diameter of the electrical ablation devices 20, 100 may be sized to fit within a working channel of an endoscope and in other embodiments the outside diameter of the electrical ablation devices 20, 100 may be sized to fit within a hollow outer sleeve, or trocar, for example.

FIG. 3 illustrates one embodiment of the electrical ablation system 10 shown in FIGS. 1 and 2A-D in use to treat undesirable tissue 48 located on the surface of the liver 50. The undesirable tissue 48 may be representative of a variety of diseased tissues, cancers, tumors, masses, lesions, abnormal tissue growths, for example. In use, the electrical ablation device 20 may be introduced into or proximate the tissue treatment region through a port 52 of a trocar 54. The trocar 54 is introduced into the patient via a small incision 59 formed in the skin 56. The endoscope 12 may be introduced into the patient trans-anally through the colon, trans-orally down the esophagus and through the stomach using translumenal techniques, or through a small incision or keyhole formed through the patient's abdominal wall (e.g., the peritoneal wall). The endoscope 12 may be employed to guide and locate the distal end of the electrical ablation device 20 into or proximate the undesirable tissue 48. Prior to introducing the flexible shaft 22 through the trocar 54, the sheath 26 is slid over the flexible shaft 22 in a direction toward the distal end thereof to cover the electrodes 24a,b (as shown in FIG. 2A) until the distal end of the electrical ablation device 20 reaches the undesirable tissue 48.

Once the electrical ablation device 20 has been suitably introduced into or proximate the undesirable tissue 48, the sheath 26 is retracted to expose the electrodes 24a,b (as shown in FIG. 2B) to treat the undesirable tissue 48. To ablate the undesirable tissue 48, the operator initially may locate the first electrode 24a at a first position 58a and the second electrode 24b at a second position 60 using endoscopic visualization and maintaining the undesirable tissue 48 within the field of view of the flexible endoscope 12. The first position 58a may be near a perimeter edge of the undesirable tissue 48. Once the electrodes 24a,b are located into or proximate the undesirable tissue 48, the electrodes 24a,b are energized with irreversible electroporation pulses to create a first necrotic zone 65a. For example, once the first and second electrodes 24a,b are located in the desired positions 60 and 58a, the undesirable tissue 48 may be exposed to an electric field generated by energizing the first and second electrodes 24a,b with the energy source 14. The electric field may have a magnitude, frequency, and pulse length suitable to induce irreversible electroporation in the undesirable tissue 48 within the first necrotic zone 65a. The size of the necrotic zone is substantially dependent on the size and separation of the electrodes 24a,b, as previously discussed. The treatment time is defined as the time that the electrodes 24a,b are activated or energized to generate the electric pulses suitable for inducing irreversible electroporation in the undesirable tissue 48.

This procedure may be repeated to destroy relatively larger portions of the undesirable tissue 48. The position 60 may be taken as a pivot point about which the first electrode 24a may be rotated in an arc of radius “r,” the distance between the first and second electrodes 24a,b. Prior to rotating about the second electrode 24b, the first electrode 24a is retracted by pulling on the slide member 30 (FIGS. 1 and 2A-D) in a direction toward the proximal end and rotating the electrical ablation device 20 about the pivot point formed at position 60 by the second electrode 24b. Once the first electrode 24a is rotated to a second position 58b, it is advanced to engage the undesirable tissue 48 at point 58b by pushing on the slide member 30 in a direction towards the distal end. A second necrotic zone 65b is formed upon energizing the first and second electrodes 24a,b. A third necrotic zone 65c is formed by retracting the first electrode 24a, pivoting about pivot point 60 and rotating the first electrode 24a to a new location, advancing the first electrode 24a into the undesirable tissue 48 and energizing the first and second electrodes 24a,b. This process may be repeated as often as necessary to create any number of necrotic zones 65p, where p is any positive integer, within multiple circular areas of radius “r,” for example, that is suitable to ablate the entire undesirable tissue 48 region. At anytime, the surgeon or clinician can reposition the first and second electrodes 24a,b and begin the process anew. In other embodiments, the electrical ablation device 100 comprising multiple needle electrodes 124a-m described with reference to FIG. 2E may be employed to treat the undesirable tissue 48. Those skilled in the art will appreciate that similar techniques may be employed to ablate any other undesirable tissues that may be accessible trans-anally through the colon, and/or orally through the esophagus and the stomach using translumenal access techniques. Therefore, the embodiments are not limited in this context.

In various embodiments, as outlined above, a surgical instrument can comprise a first electrode and a second electrode, wherein at least one the first and second electrodes can be operably coupled to a power source. In certain embodiments, as also outlined above, a first electrode can be operably coupled with a positive terminal of a voltage source and the second electrode can be operably coupled with a negative terminal of the voltage source, for example. In at least one embodiment, the first and second electrodes can comprise columnar, or point, electrodes which can be inserted into the tissue of a patient. In various circumstances, a voltage potential can be applied to the two electrodes such that a magnetic field can be created therebetween in order to treat the tissue positioned intermediate the electrodes. In some circumstances, the voltage potential may be sufficient to permit current to flow between the electrodes. In any event, the tissue that can be treated with such devices may be limited to, or at least substantially limited to, a line of tissue positioned intermediate the two electrodes. Various devices are disclosed in commonly-owned co-pending U.S. patent application Ser. No. 12/352,375, entitled ELECTRICAL ABLATION DEVICES, which was filed on Jan. 12, 2009, the entire disclosure of which is incorporated by reference herein. While such devices may be suitable for their intended purposes, other devices disclosed herein can provide different tissue treatment regions which can provide various advantages.

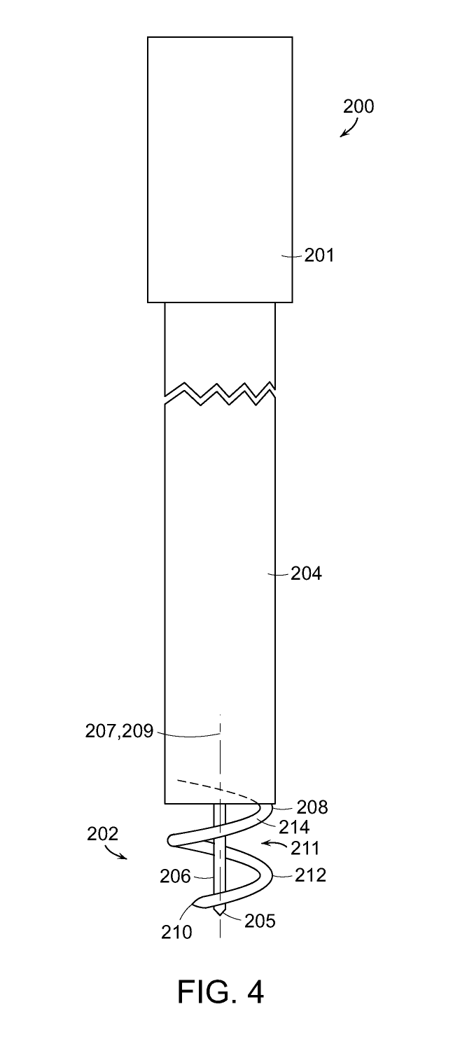

In various embodiments, referring now to FIG. 4, a surgical instrument 200 can comprise a handle 201, an end effector 202, and a shaft 204 extending between the handle and the end effector 202. Surgical instrument 200 can further comprise one or more electrodes, such as first electrode 206 and second electrode 208, for example, which can be utilized to treat tissue. In at least one embodiment, referring to FIGS. 4 and 5, the first electrode 206 can comprise a columnar electrode having a straight, or at least substantially straight, configuration. The first electrode 206 can be comprised of an electrically conductive material, such as stainless steel, for example, and can be configured to extend along a longitudinal axis, such as axis 207, for example. In various embodiments, the first electrode 206 can comprise a solid, circular, or at least substantially circular, cross-section.

In various embodiments, referring again to FIGS. 4 and 5, the second electrode 208 can comprise a spiral coil, such as spiral coil portion 211, for example, wherein the spiral coil can be formed from a single continuous wire or a plurality of wires attached in an end-to-end arrangement, for example. In either event, such electrodes, and/or similar electrodes, can be referred to as comprising a “wire”. In at least one embodiment, the wire can be comprised of a conductive material, such as stainless steel, for example, and, in various embodiments, the wire can have a solid, circular, or at least substantially circular, cross-section. In certain embodiments, the wire can be bent into a helical configuration about a central axis, such as longitudinal axis 209, for example, wherein axis 209 can be collinear with, or aligned with respect to, axis 207 of first electrode 206. More particularly, in at least one embodiment, axis 209 can be aligned with axis 207 such that first electrode 206 is positioned in the center of, or in at least substantially the center of, the spiral coil 211 of second electrode 208. In other embodiments, axis 209 can be offset from axis 207. In at least one such embodiment, axis 207 and axis 209 can be parallel, or at least substantially parallel, to one another while, in other embodiments, axis 207 and axis 209 can be skew with one another. In various embodiments, the spiral coil 211 of second electrode 208 can comprise a plurality of rings, or loops, which are elongated longitudinally along axis 209. In various circumstances, a single ring, or loop, can comprise one full revolution of the wire about axis 209. Such a ring, or loop, can begin at a first longitudinal position with respect to axis 209 and spiral about axis 209 such that the loop is completed at a second, or different, longitudinal position with respect to axis 209.

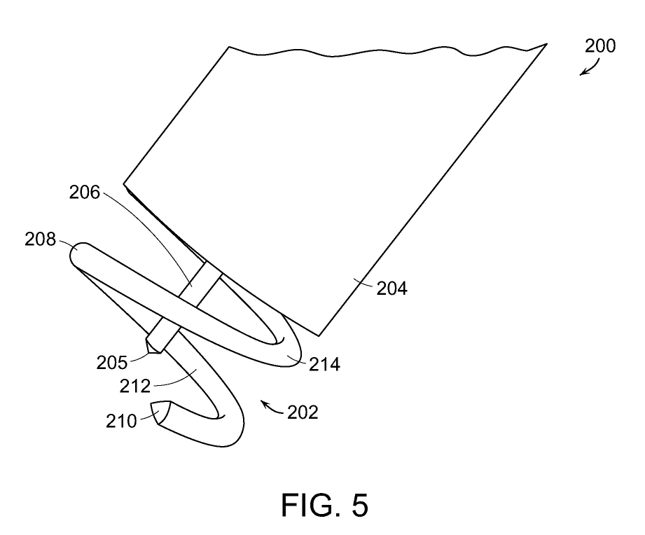

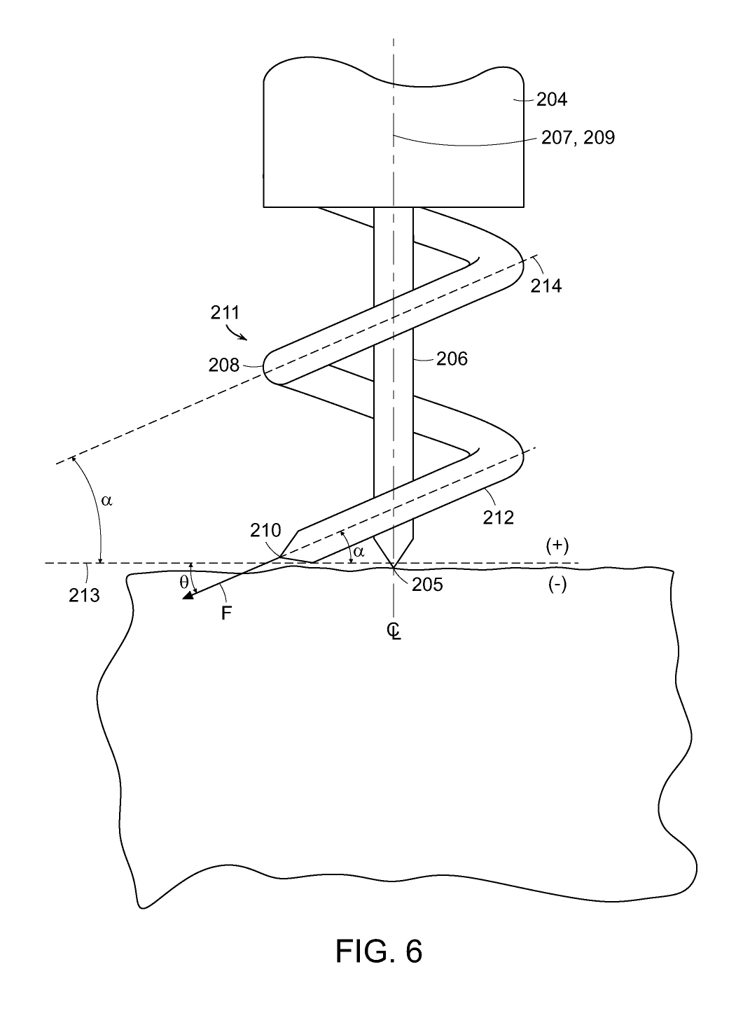

In use, further to the above, the end effector 202 of surgical instrument 200 can be positioned against the tissue to be treated, as illustrated in FIG. 6, and inserted into the tissue, as illustrated in FIG. 7. In various embodiments, at least a portion of the end effector 202 can be rotated such that second electrode 208 is rotated, or screwed, into the tissue. In at least one embodiment, second electrode 208 can comprise a distal end 210 which can be configured to penetrate the tissue. In certain embodiments, distal end 210 can comprise a sharp point and/or a conical profile ending in a sharp point. In certain circumstances, the distal end 210 can be configured to enter into the tissue when end effector 202, or at least second electrode 208, is rotated relative to the tissue. Once distal end 210 has entered into the tissue, further rotation of second electrode 208 can drive distal end 210 deeper into the tissue. More particularly, one or more of the rings of the spiral coil 211, such as end ring 212, for example, can be inclined or oriented at an angle such that the rotational force applied to the end effector 202, or second electrode 208, can cause the end ring 212 and distal end 210 to move circumferentially about an axis of rotation and, at the same time, downwardly into the tissue. Generally speaking, the term downwardly can comprise any direction which is in the direction of the tissue being treated, for example. In at least one embodiment, referring again to FIG. 7, the term downwardly can comprise a direction which is oriented to the negative (−) side of plane 213. In various embodiments, plane 213 can be orthogonal, or at least substantially orthogonal, to axis 207 of first electrode 206 and/or axis 209 of second electrode 208.

In various embodiments, further to the above, a rotational force and/or downward force can be applied to the handle of surgical instrument 200, for example, wherein such forces can be transmitted into the distal end 210 of second electrode 208 such that the distal end 210 can direct an insertion force, F, into the tissue. In certain embodiments, as described in greater detail below, a surgical instrument can comprise one or more motors for applying a rotational force and/or a downward force to second electrode 208. In any event, referring again to FIG. 7, the insertion force F can be represented by a vector, wherein the direction of the vector can be represented by an insertion angle, θ, measured from plane 213, for example. In various embodiments, the insertion angle θ can be approximately 5 degrees, approximately 10 degrees, approximately 15 degrees, approximately 20 degrees, approximately 25 degrees, approximately 30 degrees, approximately 35 degrees, approximately 40 degrees, and/or approximately 45 degrees, for example. In certain embodiments, the insertion angle θ can be between approximately 1 degree and approximately 10 degrees, between approximately 10 degrees and approximately 20 degrees, between approximately 20 degrees and approximately 30 degrees, between approximately 30 degrees and approximately 40 degrees, and/or between approximately 40 degrees and approximately 50 degrees, for example.

In various embodiments, further to the above, each ring of the spiral coil 211 can be oriented at the same, or at least substantially the same, angle of inclination. The angle of inclination of a ring can be referred to as the pitch angle α of the ring, wherein the pitch angle α can convey the pitch of the ring with respect to a datum, such as plane 213, for example. In at least one embodiment, for example, end ring 212 can be inclined at a 10 degree pitch angle, a first adjacent ring 214 can be inclined at a 10 degree pitch angle, and a second adjacent ring can also be inclined at a 10 degree pitch angle, for example. In various circumstances, such a spiral coil may be referred to as having a constant pitch, or constant pitch angle α. In any event, at least with regard to the embodiment of FIGS. 6 and 7, the reader will note that the pitch angle α of end ring 212 can be the same, or at least substantially the same, as the insertion angle θ of distal end 210. In various embodiments, the pitch angle α can be approximately 5 degrees, approximately 10 degrees, approximately 15 degrees, approximately 20 degrees, approximately 25 degrees, approximately 30 degrees, approximately 35 degrees, approximately 40 degrees, and/or approximately 45 degrees, for example. In certain embodiments, the pitch angle α can be between approximately 1 degree and approximately 10 degrees, between approximately 10 degrees and approximately 20 degrees, between approximately 20 degrees and approximately 30 degrees, between approximately 30 degrees and approximately 40 degrees, and/or between approximately 40 degrees and approximately 50 degrees, for example.

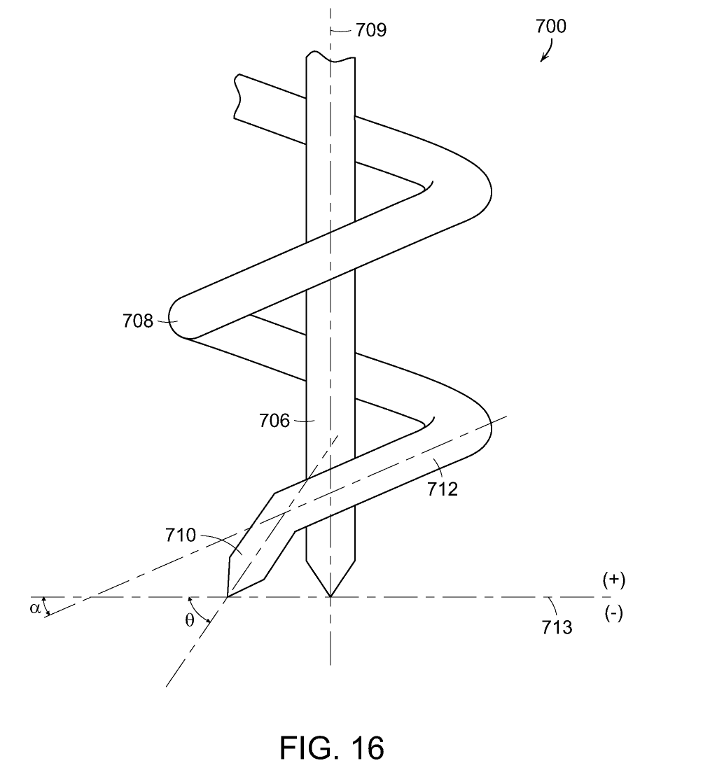

Other various embodiments are envisioned wherein the pitch angle α of a spiral coil end ring is not the same as the insertion angle θ of the spiral coil distal end. In at least one embodiment, referring now to FIG. 16, a surgical instrument 700 can comprise a first electrode 706 and a second electrode 708, wherein the second electrode 708 can, similar to the above, comprise a wire having a spiral coil configuration, and wherein the spiral coil can comprise a plurality of rings which are elongated longitudinally along axis 709. As illustrated in FIG. 16, the distal end 710 of the spiral coil can extend at an angle, i.e., insertion angle θ, with respect to datum 713 which is different than the angle, i.e., pitch angle α, of end ring 712. In at least one such embodiment, the pitch angle α of end ring 712 can be approximately 10 degrees, for example, while the insertion angle θ of distal end 710 can be approximately 30 degrees, for example. In various embodiments, the insertion angle θ of distal end 710 can be larger than the pitch angle α of end ring 712 while, in other embodiments, the insertion angle θ of distal end 710 can be less than the pitch angle α of end ring 712. In embodiments where the insertion angle θ of distal end 710 is larger than the pitch angle α of end ring 712, the distal end 710 can be driven deeper into the patient's tissue with each turn of the end effector 702, and/or second electrode 708. More particularly, in at least one embodiment, the larger insertion angle θ can, owing to its steeper angle, direct a larger portion of the force, or forces, being applied to the surgical instrument to push distal end 710 deeper into the tissue.

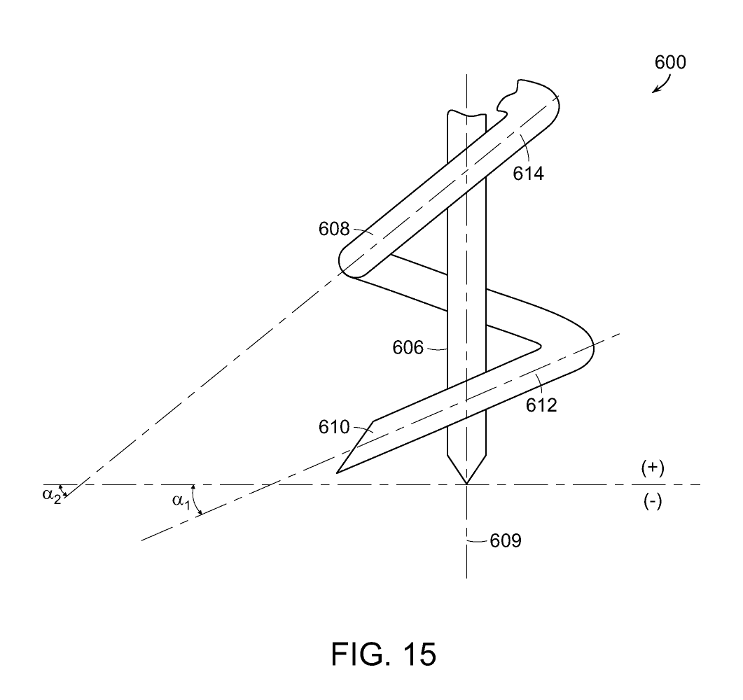

In various embodiments, referring now to FIG. 15, a surgical instrument, such as surgical instrument 600, for example, can comprise a first electrode 606 and a second electrode 608, wherein the second electrode 608 can, similar to the above, comprise a wire having a spiral coil configuration. In at least one embodiment, the spiral coil can comprise a plurality of rings which are elongated longitudinally along axis 609. In various embodiments, the rings of the spiral coil can be oriented at different angles of inclination, or pitch angles a. In at least one such embodiment, the pitch angle α₁ of end ring 612 can be approximately 10 degrees, for example, the pitch angle α₂ of first adjacent ring 614 can be approximately 15 degrees, for example, and the pitch angle α₃ of a second adjacent ring can be approximately 20 degrees, for example. Such embodiments can be referred to as having a non-constant, or variable, pitch, or pitch angle. In embodiments where the most distal ring, such as end ring 612, for example, has a smaller, or “flatter”, pitch angle than more proximal rings, such as ring 614, for example, the force, or forces, required to insert ring 612 into the tissue would be less than the force, or forces, required to insert ring 614 into the tissue. Stated another way, the smaller, or “flatter”, pitch angle of ring 612 can allow the spiral coil to be rotated into the tissue with a smaller initial force as compared to the force required to further rotate the spiral coil into the tissue once ring 614 enters into the tissue. In various circumstances, the smaller or flatter pitch angle of ring 612, while it may be less mechanically efficient than the larger, or “steeper”, pitch angle of ring 614, may be able to direct a larger proportion of the rotational force, or forces, applied to the surgical instrument 600, or second electrode 608, into rotating the distal end 610 within the tissue. Such embodiments can facilitate the initial insertion of second electrode 608 into the tissue.

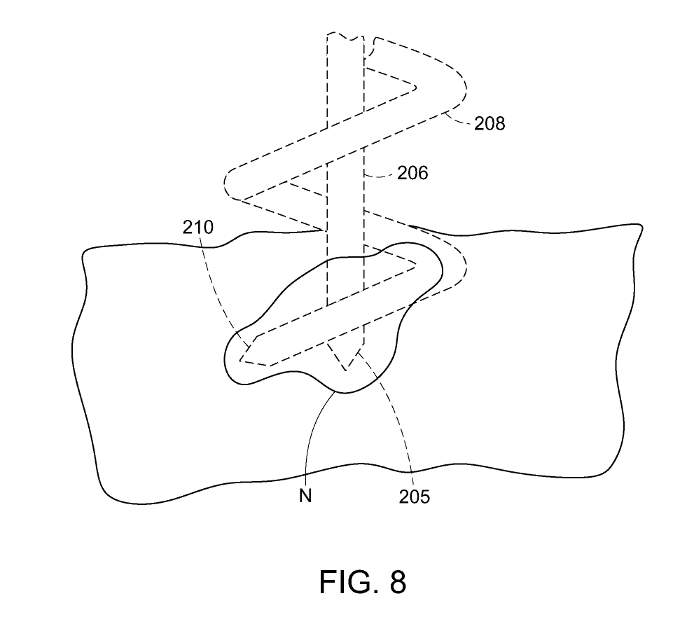

In various embodiments, referring again to FIGS. 6 and 7, the distal end 210 of second electrode 208 can be positioned against the tissue of a patient before it is inserted into the tissue. In certain embodiments, further to the above, the distal end 210 can lie within plane 213 such that, when distal end 210 has been positioned against the tissue, plane 213 can be aligned with the surface of the tissue. In at least one embodiment, the distal end 205 of first electrode 206 can also lie within, or can be positioned adjacent to, plane 213. When the distal end 210 of second electrode 208 is rotated, or advanced, into the tissue, as described above, the distal end 205 of first electrode 206 can also be advanced into the tissue. In at least one such embodiment, the distal end 205 can comprise a sharp point and/or a conical profile ending in a sharp point, for example, wherein, as distal end 210 of second electrode 208 is advanced downwardly into the tissue, the distal end 205 can also be advanced downwardly into the tissue. In various alternative embodiments, referring to FIG. 9, a surgical instrument, such as surgical instrument 300, for example, can comprise a center electrode 306 and a helical electrode 308, wherein the distal end 305 of center electrode 306 can extend beyond the distal end 310 of helical electrode 308. In at least one such embodiment, the distal end 305 of center electrode 306 can be positioned against the targeted tissue wherein the distal end 305 can be pushed downwardly into the tissue in order to place distal end 310 of helical electrode 308 in contact with the tissue. In at least one such embodiment, the center electrode 306 can be pre-inserted into the tissue before the helical electrode 308 is inserted, or rotated, into the tissue. In various other embodiments, a distal end of a first electrode, such as a columnar electrode, for example, can be recessed with respect to a distal end of a second electrode, such as a helical electrode, for example. In at least one embodiment, the distal end of the helical electrode can define, or lie within, a plane wherein the distal end of the second electrode can be recessed with respect to the plane. In use, the helical electrode can be positioned against tissue such that the first electrode is not engaged with the tissue. As the helical electrode is rotated into the tissue, the first electrode can contact and/or penetrate the tissue.

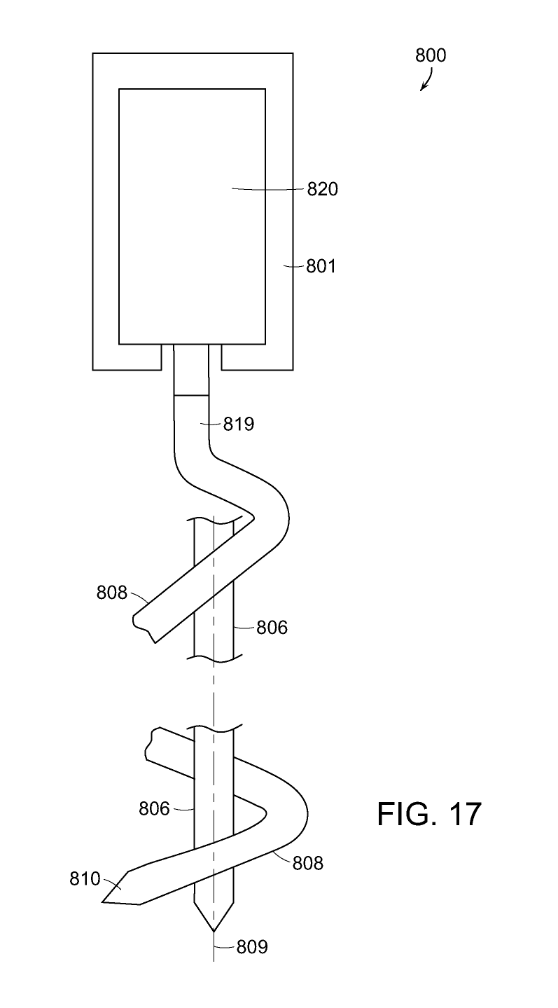

In various embodiments, referring to FIG. 4 once again, the proximal ends of electrode 206 and electrode 208, and/or any other suitable portions thereof, can be mounted to, and/or otherwise secured to, surgical instrument 200 such that a downward force and/or a rotational force applied to surgical instrument 200 can be transmitted to first electrode 206 and/or second electrode 208. In various circumstances, such forces can be applied to surgical instrument 200 by the surgeon. In certain embodiments, referring now to FIG. 17, a surgical instrument, such as surgical instrument 800, for example, can comprise a motor, such as motor 820, for example, which can be configured to rotate spiral coil electrode 808 into tissue. More particularly, in at least one embodiment, the electrode 808 can comprise a proximal end 819 which can be operably engaged with motor 820 such that, when motor 820 is actuated, motor 820 can rotate electrode 808 in a first direction. Owing to the rotation of electrode 808 in the first direction, the distal end 810 of electrode 808 can penetrate and enter into the targeted tissue. Correspondingly, the motor 820 can be configured to rotate electrode 808 in a second, or opposite, direction in order to withdraw the electrode 808 from the tissue. In at least one embodiment, the motor 820 can be configured to rotate electrode 808 about an axis 809. In certain embodiments, the surgical instrument 800 can further comprise a straight, or at least substantially straight, columnar electrode 806, wherein the motor 820 can be configured to rotate spiral electrode 808 about columnar electrode 806, for example. In at least one embodiment, the motor 820 can be positioned within and/or secured within a handle 801, for example, of the surgical instrument 800. In use, a surgeon can grasp the handle 801 of surgical instrument 800 and place the end effector 802 of surgical instrument 800 against the targeted tissue. Once satisfied with the positions of electrodes 806 and 808, the surgeon can operate a switch on the handle, for example, to operate the motor 820. In other circumstances, the surgeon can operate the motor 820 before it is contacted with the tissue.

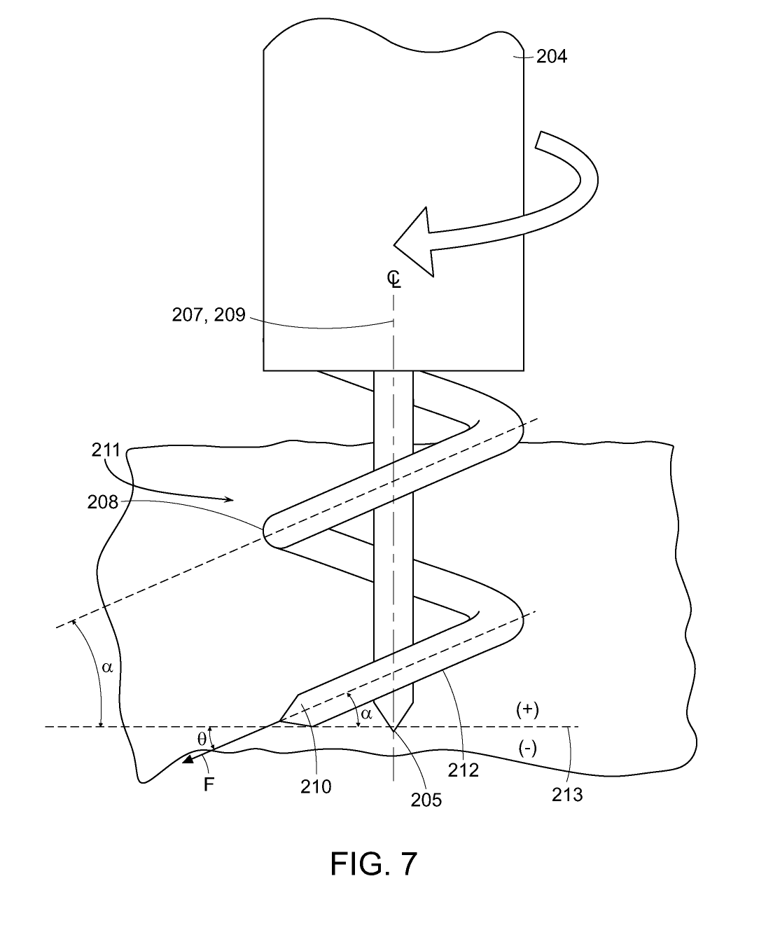

Referring again to the embodiment of FIG. 4, after first electrode 206 and second electrode 208 have been suitably positioned within and/or relative to the tissue to be treated, referring now to FIG. 8, a voltage differential can be created between the electrodes 206 and 208 such that current can flow through the tissue between the first electrode 206 and the second electrode 208. In at least one embodiment where the voltage potential of the first electrode 206 is greater than the second electrode 208, current can flow radially outwardly from first electrode 206 to second electrode 208. More particularly, as illustrated in FIG. 4, the spiral coil 211 of second electrode 208 can surround, or encompass, first electrode 206 such that, when current flows from first electrode 206 to second electrode 208, the current can flow away from the columnar first electrode 206 to a circular, or at least substantially circular, perimeter defined by second electrode 208. In various embodiments, the current flowing between the first electrode 206 and the second electrode 208 may take the shortest path therebetween. In at least one circumstance, as a result, the current may flow in a substantially planar field between the first electrode 206 and the second electrode 208. In other circumstances, the second electrode 208 can define a cylindrical, or at least substantially cylindrical, perimeter surrounding the first electrode 206 such that, as a result, the current may flow in a three-dimensional field intermediate first electrode 206 and second electrode 208, for example. In various embodiments, the spiral coil 211 can completely encompass the first electrode 206 such that the current flow can be contained, or at least substantially contained, within the second electrode 208. In various other embodiments, the voltage potential of the second electrode 208 can be greater than the first electrode 206 wherein current can flow from the second electrode 208 to the first electrode 206. In at least one such embodiment, the current can flow radially inwardly from second electrode 208 to first electrode 206.

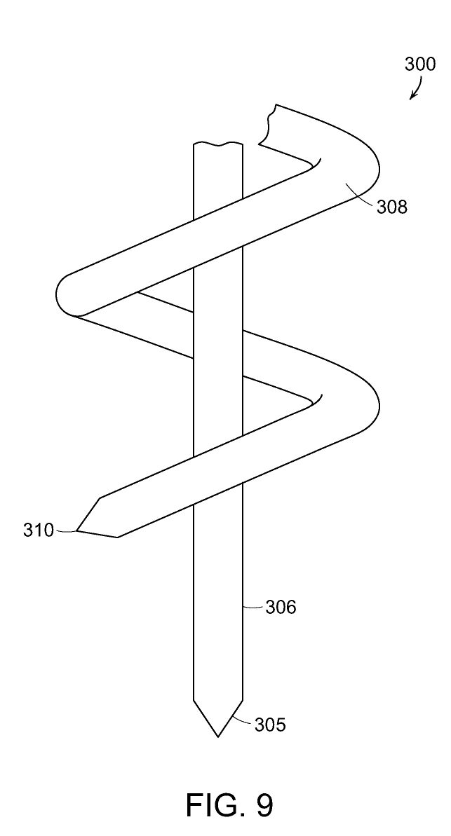

In various embodiments, further to the above, the current flowing between first electrode 206 and second electrode 208 can ablate the tissue positioned within or encompassed by second electrode 208. In certain embodiments, the current may also ablate the tissue positioned underneath and/or surrounding the second electrode 208. Referring to FIG. 8, the current can ablate a volume of tissue “N” proximal to the first and second electrodes, wherein volume of tissue N can represent the region treated by the surgical instrument. In various circumstances, the current can ablate the tissue positioned intermediate first electrode 206 and second electrode 208 and, in addition, the tissue surrounding or encompassing the outer perimeter of second electrode 208. In various circumstances, the volume of tissue N can extend around the distal end 210 of second electrode 208 and the distal end 205 of first electrode 206. As illustrated in FIG. 10, in various other embodiments, the volume of tissue N′ can extend distally from the end of helical electrode 308 along the distal end 305 such that the volume of tissue N′ extends deeper into the tissue.

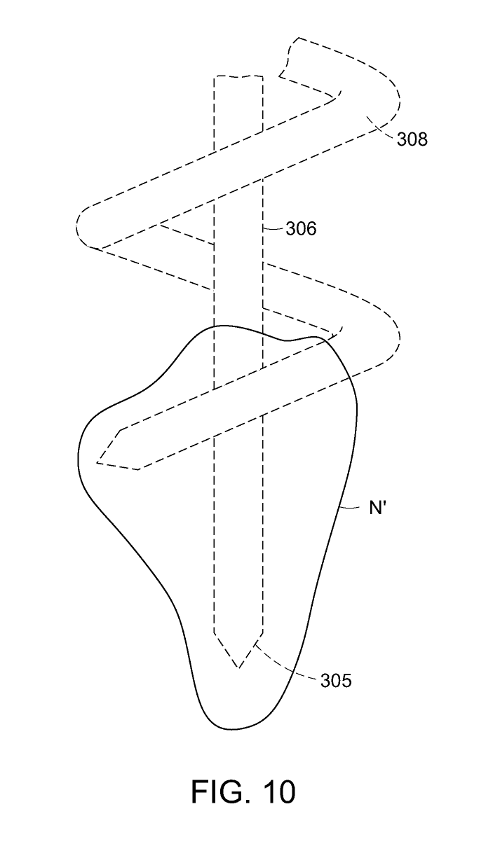

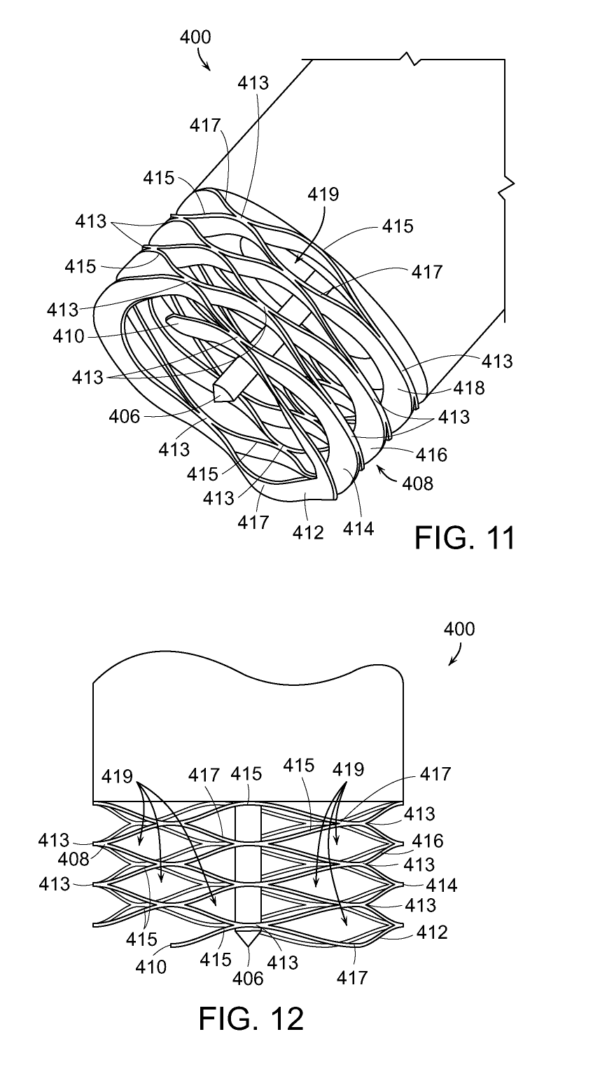

In various embodiments, referring now to FIG. 11, a surgical instrument, such as surgical instrument 400, for example, can comprise a first electrode, such as columnar electrode 406, for example, and a second electrode, such as electrode 408, for example, wherein electrode 408 can at least partially surround or encompass the first electrode 406. In at least one embodiment, the second electrode 408 can comprise a plurality of annular, or at least substantially annular, members, or rings, which can be affixed to one another such that current can flow from one annular member to another. In various embodiments, the second electrode can comprise a continuous ribbon wound around electrode 406 in a spiral fashion wherein revolutions of the continuous ribbon can be referred to as annular members or rings. In either event, the second electrode 408 can comprise an end annular member, or ring, 412 and a first adjacent annular member, or ring, 414, wherein at least portions of the first adjacent annular member 414 can be affixed to end annular member 412. More particularly, in at least one embodiment, annular member 414 can be welded to end annular member 412 at one or more locations 413. In embodiments where second electrode 408 comprises a continuous ribbon, the ribbon can comprise pinch points bent into it which can, in some embodiments, be connected in at least some fashion in order to prevent tissue from slipping should the probe position change. In certain embodiments, the annular members can be planar, or at least substantially planar, while, in other embodiments, the annular members, referring to FIG. 12, can comprise a wave-like configuration which can extend above and/or below a central plane. More particularly, in at least one embodiment, the annular members 412, 414, 416, and/or 418, for example, can comprise one or more peaks, crests, or ridges, 415 and/or one or more valleys 417, wherein the peaks 415 of a first annular member can be aligned, or at least substantially aligned, with the valleys 417 of a second, or adjacent, annular member. Correspondingly, the valleys 417 of the first annular member can be aligned, or at least substantially aligned, with the peaks 415 of the second annular member. Owing to such an arrangement, various gaps, such as gaps 419, for example, can be defined between two adjacent annular members. When the distal end 410 of second electrode 408 is inserted into tissue, the tissue can enter into gaps 419 and, when second electrode 408 is rotated within the tissue, various portions of the annular members can pinch or compress the tissue. More particularly, in at least one embodiment, the annular members defining gaps 419 can converge inwardly toward one another at locations adjacent to welded locations 413 in order to comprise pinch points which can pinch, or compress, tissue when the second electrode 408 is rotated within the tissue, for example. Stated another way, the wave-like annular members can be configured to cooperate with one another to bite into the tissue and assist in securing, or anchoring, the distal end 410 of second electrode 408 within the tissue.

In various embodiments, further to the above, each annular member, or ring, of second electrode 408 can comprise a rectangular cross-section, wherein the width of the cross-section can be larger than the height. In at least one embodiment, the width can be approximately 5 times as large as the height, while, in other embodiments, the width can be approximately 10 times as large as the height, for example. In any event, each annular member can comprise a ring wherein, owing to the peaks 415 and valleys 417 therein, can resemble a wave spring, for example. In certain embodiments, the annular members of second electrode 408 can comprise a lattice which, in at least some embodiments, can be compressed axially. In various embodiments, referring again to FIGS. 11 and 12, the end annular member, or ring, 412 can comprise a distal end 410 which can be configured to penetrate tissue. In at least one embodiment, the distal end 410 can comprise a spade-shaped configuration. In at least one such embodiment, the cross-section of the wire comprising second electrode 408 can be rectangular wherein, at the distal end 412, one or more of the sides of the rectangular wire can converge, or taper, inwardly toward one another to comprise a point, or spade, configuration. In various embodiments, two opposing sides of the rectangular wire can converge inwardly. In other embodiments, all four sides of the rectangular wire can converge inwardly. In any event, such a distal end 410 can facilitate the insertion of second electrode 408 into the tissue.

In various embodiments, as described above, a surgical instrument can comprise a first electrode and a second electrode, wherein the second electrode can surround or encompass at least a portion of the first electrode. With regard to surgical instrument 200, as illustrated in FIGS. 4 and 5, the spiral coil 211 of the second electrode 208 can surround a columnar portion of first electrode 206. In various embodiments, the spiral coil 211 can surround or encompass the entire perimeter of first electrode 206 along the entire length, or at least a portion of the length, of first electrode 206. In various other embodiments, a surgical instrument can comprise an electrode which may only partially surround or partially encompass the perimeter of another electrode. In at least one embodiment, an arcuate electrode of a surgical instrument may be positioned relative to another electrode, wherein the arcuate electrode can comprise a curved configuration which extends around less than all of the sides of the other electrode. In certain embodiments, an arcuate electrode can comprise a crescent shape defined by one or more radiuses of curvature. In various embodiments, a surgical instrument can comprise three or more electrodes. In at least one embodiment, a surgical instrument can comprise a center electrode, a first arcuate electrode positioned to one side of the center electrode, and a second arcuate electrode positioned to another side of the center electrode, wherein the first arcuate electrode and the second arcuate electrode can each be configured to surround or encompass at least a portion of the perimeter of the center electrode. In at least one such embodiment, the center electrode can comprise a straight, or at least substantially straight, columnar electrode positioned along an axis while, in other embodiments, the center electrode can comprise any suitable configuration, such as configurations which are symmetrical with respect to an axis and/or configurations which are asymmetrical with respect to an axis. In at least one embodiment, the center electrode can be closer to a first electrode than a second electrode at one point along its length while closer to the second electrode than the first electrode at another point along its length.

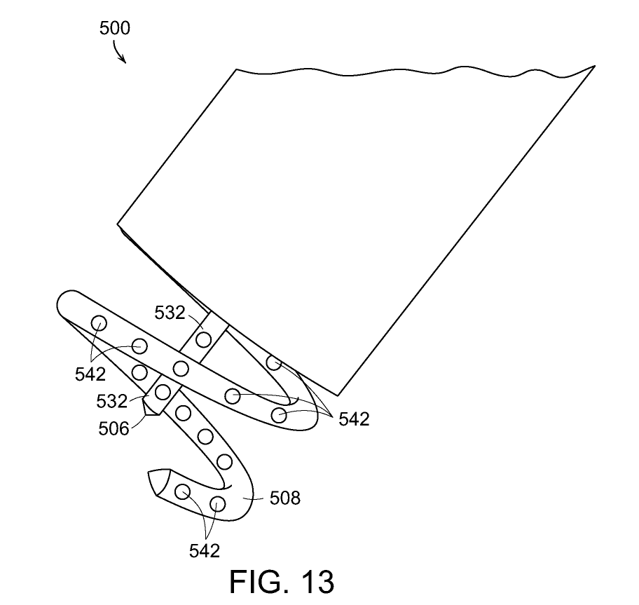

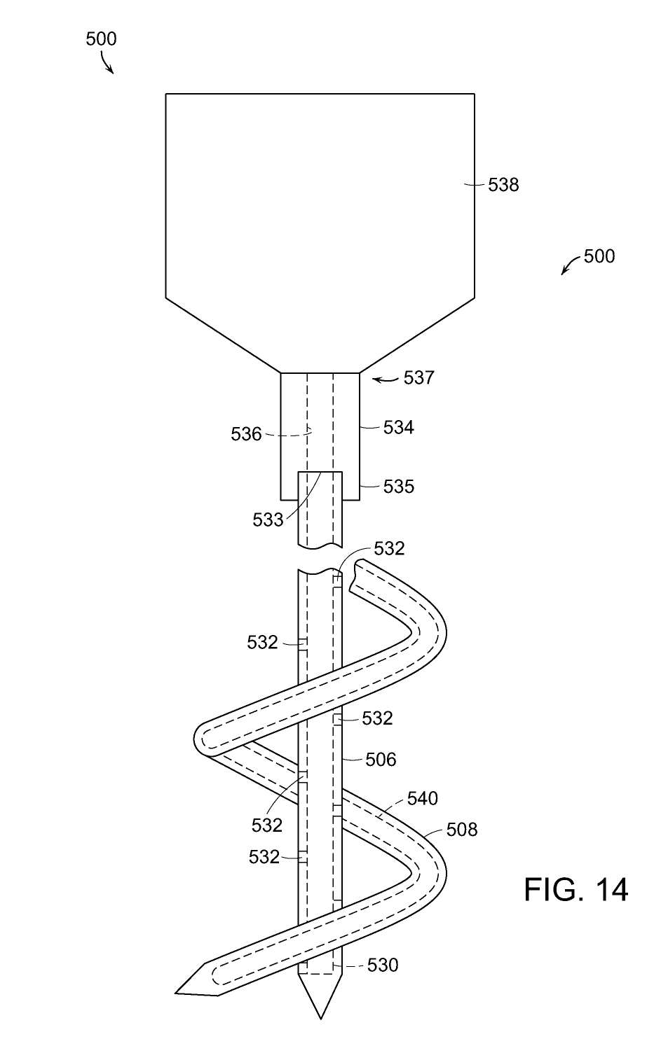

In various embodiments, referring now to FIGS. 13 and 14, a surgical instrument, such as surgical instrument 500, for example, can comprise an electrode, such as first electrode 506, for example, wherein the first electrode 506 can comprise at least one passageway, such as passageway 530, for example, defined therein. In certain embodiments, passageway 530 can be configured such that a fluid can be conveyed therethrough. In use, in at least embodiment, the first electrode 506 can be positioned against and/or inserted into the tissue to be treated, wherein, similar to the above, a voltage potential can be applied to the first electrode 506 such that current can flow between the first electrode 506 and another electrode, such as second electrode 508, for example. In various circumstances, also similar to the above, the current can ablate the tissue positioned between and/or relative to the first electrode 506 and the second electrode 508. Before and/or after the tissue has been ablated and/or otherwise treated, in various circumstances, a therapeutic liquid, saline, and/or fluid containing a toxic molecule, for example, can be passed through the passageway 530 in order to treat the tissue. In various circumstances, saline can change the conductivity of the surrounding tissue. In certain circumstances, a fluid can comprise a toxic molecule such as Bleomycin, for example. In any event, a fluid can be applied directly to the tissue being treated without having to remove surgical instrument 500 from the tissue and/or without having to insert an additional surgical instrument into the tissue to supply the therapeutic fluid. In various embodiments, referring now to FIG. 14, the first electrode 506 can comprise one or more openings or apertures, such as apertures 532, for example, which can be in fluid communication with passageway 530. As a result of apertures 532 being in fluid communication with passageway 530, the fluid within passageway 530 can flow through apertures 532 and into the tissue. In various embodiments, the first electrode 506 can comprise a plurality of apertures 532 positioned along the length thereof, although any suitable arrangement can be used. In at least the illustrated embodiment of FIG. 14, an axial array of apertures 532 can permit the fluid to exit passageway 530 at multiple locations, or depths, within the tissue.

In various embodiments, further to the above, the second electrode 506 can comprise a proximal opening which can allow the therapeutic fluid to be inserted into passageway 530. In various circumstances, an end, or needle, of a syringe can be inserted into the proximal opening such that the syringe can be operated to release the therapeutic fluid into passageway 530. In certain circumstances, depending on the orientation of surgical instrument 500, the fluid can flow to the distal apertures 532 with the assistance of a gravitational force. In some circumstances, the syringe can be operated to project the fluid into passageway 530 with sufficient force and momentum such that the fluid can overcome a gravitational force to reach the distal apertures 532. In various embodiments, referring now to FIG. 14, surgical instrument 500 can further comprise a delivery tube, such as delivery tube 534, for example, which can comprise a passageway 536 in fluid communication with passageway 530 of first electrode 506. In certain embodiments, the delivery tube 534 can comprise a distal end 535 in fluid communication with a proximal aperture 533 in first electrode 506 and, in addition, a proximal end 537 comprising a syringe housing 538 which can be configured to receive and/or can otherwise be operatively engaged with a syringe. In various embodiments, the delivery tube can be comprised of a sufficiently non-conductive material, such as Teflon, for example, such that the syringe housing 538 can be electrically insulated from first electrode 506. In certain embodiments, the surgical instrument 500 can comprise a reservoir configured to store one or more therapeutic fluids therein and, in addition, at least one valve which can be configured to retain the fluid in the reservoir until the valve is operated to release the fluid.

In various embodiments, the second electrode 508 of surgical instrument 500 can comprise a passageway defined therein which can be configured to deliver a therapeutic fluid into the tissue. In at least one embodiment, referring again to FIGS. 7 and 8, the second electrode 508 can comprise a passageway 540 extending therethrough and one or more openings, or apertures, such as apertures 542, for example, in fluid communication with passageway 540 such that fluid flowing through passageway 540 can flow into the tissue through apertures 542. In various embodiments, the surgical instrument 500 can further comprise a delivery tube in fluid communication with syringe housing 538. In various embodiments, the delivery tube can be comprised of a sufficiently non-conductive material, such as Teflon, for example, such that the syringe housing 538 can be electrically insulated from second electrode 508. In various embodiments, a syringe can be operably coupled with syringe housing 538 such that, when the syringe is operated to dispense the therapeutic fluid contained therein, the fluid can be delivered to the tissue through first electrode 506 and second electrode 508. In at least one embodiment, although not illustrated, the surgical instrument 500 can comprise a valve which can be configured to allow a surgeon to selectively permit the fluid to flow through only one of the electrodes 506 and 508.

In various other embodiments, further to the above, a surgical instrument can comprise a first reservoir in fluid communication with a first delivery tube and a first electrode and, in addition, a second reservoir in fluid communication with a second delivery tube and a second electrode. In at least one such embodiment, the surgical instrument can further comprise a first valve configured to be selectively operated to release a first fluid from the first reservoir and, in addition, a second valve configured to be selectively operated to release a second fluid from the second reservoir. In various circumstances, such surgical instruments can allow a surgeon to release the first and second fluids sequentially and/or simultaneously. In some circumstances, certain therapeutic advantages can be obtained by maintaining the first and second fluids in separate containers, or systems, prior to their insertion into the tissue. Similar advantages can be obtained when a surgical instrument has a first syringe housing configured to receive a first syringe and a second syringe housing configured to receive a second syringe. In at least one such embodiment, the syringes can be operated sequentially and/or simultaneously. In any event, the surgical instruments can be configured such that the fluids do not mix until they are in the tissue.

Various embodiments are described above in which a surgical instrument has a first electrode and a second electrode, wherein both of the first and second electrodes can be configured to convey a fluid to a surgical site. Various other embodiments are envisioned in which only the first electrode or the second electrode can be configured to convey a fluid to a surgical site. In at least one embodiment, the first electrode, which can be similar to first electrode 506, for example, can comprise a fluid passageway therein while the second electrode, which can be similar to the second electrode 208, for example, can be comprised of a solid wire. In other embodiments, the first electrode, which can be similar to first electrode 206, for example, can be comprised of a solid wire while the second electrode, which can be similar to the second electrode 508, for example, can comprise a fluid passageway therein. Various other embodiments are envisioned in which a surgical instrument comprises three or more electrodes wherein any suitable amount of the electrodes can comprise one or more passageways defined therein configured to deliver a therapeutic fluid to a surgical site.

In use, as described above, surgical instrument 500 can be configured to ablate or treat the tissue positioned within and/or around the second electrode 508. In various circumstances, the distal end 510 of second electrode 508 can be positioned such that it encompasses a tumor. In various embodiments, the perimeter of the second electrode 508 can encompass or surround the perimeter of the tumor, whereas the first electrode 506 can be inserted into the tumor. In some circumstances, liver tumors having a diameter of approximately 2.5 cm, or larger than 2.5 cm, for example, can occur. In certain embodiments, the diameter of the second electrode 508 can be approximately 2.5 cm, or larger than 2.5 cm, for example. In at least one embodiment, the second electrode 508 can be screwed into the tissue, as outlined above, such that the entirety, or at least the substantial entirety, of the tumor is positioned within the second electrode 508. In use, as outlined above, a therapeutic fluid can be delivered to the tissue after the tumor has been ablated, or otherwise treated, with surgical instrument 500. In certain circumstances, one or both of the electrodes 506 and 508 can be removed from the tissue before the therapeutic is dispensed. In various circumstances, a therapeutic fluid can be delivered to the tissue before the tumor is ablated and/or while the tissue is being ablated. In at least some embodiments, the therapeutic fluid can be conductive and can increase the conductivity of the tumor tissue and, when the therapeutic fluid is delivered before and/or during the activation of the electrodes, the fluid can facilitate the ablation of the tumor. In various embodiments, further to the above, a first therapeutic fluid can be delivered to the tissue before the electrodes are activated and a second therapeutic fluid can be delivered to the tissue after the electrodes have been activated. In at least one such embodiment, the first therapeutic fluid can increase the conductivity of the tumor tissue while the second therapeutic fluid can kill any remaining tumor cells in the tissue.

The embodiments of the devices described herein may be introduced inside a patient using minimally invasive or open surgical techniques. In some instances it may be advantageous to introduce the devices inside the patient using a combination of minimally invasive and open surgical techniques. Minimally invasive techniques may provide more accurate and effective access to the treatment region for diagnostic and treatment procedures. To reach internal treatment regions within the patient, the devices described herein may be inserted through natural openings of the body such as the mouth, anus, and/or vagina, for example. Minimally invasive procedures performed by the introduction of various medical devices into the patient through a natural opening of the patient are known in the art as NOTES™ procedures. Some portions of the devices may be introduced to the tissue treatment region percutaneously or through small—keyhole—incisions.

Endoscopic minimally invasive surgical and diagnostic medical procedures are used to evaluate and treat internal organs by inserting a small tube into the body. The endoscope may have a rigid or a flexible tube. A flexible endoscope may be introduced either through a natural body opening (e.g., mouth, anus, and/or vagina) or via a trocar through a relatively small—keyhole—incision incisions (usually 0.5-1.5 cm). The endoscope can be used to observe surface conditions of internal organs, including abnormal or diseased tissue such as lesions and other surface conditions and capture images for visual inspection and photography. The endoscope may be adapted and configured with working channels for introducing medical instruments to the treatment region for taking biopsies, retrieving foreign objects, and/or performing surgical procedures.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam.

In various embodiments, the devices disclosed herein, and/or the electrodes thereof, can be cleaned utilizing one or more enzymatic cleaners, detergents, and/or acids, and/or one or more steam cleaning and/or mechanical polishing processes, for example. In various embodiments, one or more of the electrodes of the devices disclosed herein can be removed from the device and replaced. In certain embodiments, one or more of the electrodes can be removed from the device, cleaned, and reattached via solder, welding, epoxy, and/or an adhesive, for example. In various embodiments, the devices disclosed herein, and/or the electrodes thereof, can be sterilized utilizing steam, gamma radiation, ethylene oxide, peroxide solution, and/or electron-beam processes, for example.

Although the various embodiments of the devices have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A surgical instrument configured to deliver electrical energy to the tissue of a patient, comprising:
a first electrode comprising a distal portion configured to contact the tissue; and
a second electrode comprising a distal portion configured to be inserted into the tissue, wherein said distal portion of said second electrode at least partially encompasses said distal portion of said first electrode.

2. The surgical instrument of claim 1, wherein said distal portion of said second electrode comprises a spiral coil, and wherein said distal portion of said first electrode is positioned within said spiral coil.

3. The surgical instrument of claim 2, wherein said spiral coil comprises a distal end configured to penetrate tissue when said spiral coil is rotated about an axis.

4. The surgical instrument of claim 2, wherein said spiral coil is defined about an axis, and wherein said distal portion of said first electrode is positioned along said axis.

5. The surgical instrument of claim 4, wherein said spiral coil is configured to be rotated about said first electrode.

6. The surgical instrument of claim 2, wherein said spiral coil comprises a distal end configured to penetrate tissue, wherein said first electrode comprises a distal end configured to penetrate tissue, and wherein said distal end of said first electrode is recessed with respect to said distal end of said spiral coil.

7. The surgical instrument of claim 2, wherein said spiral coil is comprised of a wire having a circular cross-section.

8. The surgical instrument of claim 2, wherein said spiral coil is comprised of a wire having a rectangular cross-section.

9. The surgical instrument of claim 2, wherein said spiral coil comprises a plurality of helical rings, wherein each said helical ring is inclined at a pitch angle, and wherein the pitch angle of each helical ring is the same.

10. The surgical instrument of claim 2, wherein said spiral coil comprises a first helical ring and a second helical ring, wherein said first helical ring is connected to said second helical ring, wherein said first helical ring is inclined at a first pitch angle, wherein said second helical ring is inclined at a second pitch angle, and wherein said first pitch angle is different than said second pitch angle.

11. The surgical instrument of claim 1, wherein said distal portion of said second electrode comprises a plurality of connected rings, and wherein said distal portion of said first electrode is positioned within said connected rings.

12. The surgical instrument of claim 11, wherein one of said rings comprises a distal end configured to penetrate tissue when said second electrode is rotated about an axis.

13. The surgical instrument of claim 11, wherein said connected rings are defined about an axis, and wherein said distal portion of said first electrode is positioned along said axis.

14. The surgical instrument of claim 13, wherein said connected rings are configured to be rotated about said first electrode.

15. The surgical instrument of claim 11, wherein one of said rings comprises a distal end configured to penetrate tissue, wherein said first electrode comprises a distal end configured to penetrate tissue, and wherein said distal end of said first electrode is recessed with respect to said distal end of said ring.

16. The surgical instrument of claim 11, wherein said rings are comprised of a wire having a circular cross-section.

17. The surgical instrument of claim 11, wherein said rings are comprised of a wire having a rectangular cross-section.

18. A surgical instrument configured to deliver electrical energy to the tissue of a patient, comprising:
a first electrode comprising a distal portion configured to be inserted into the tissue; and
a second electrode comprising a distal portion configured to be inserted into the tissue, wherein said distal portion of said second electrode at least partially surrounds said distal portion of said first electrode.

19. A surgical instrument configured to deliver electrical energy to the tissue of a patient, comprising:
a first electrode, comprising:
a first distal portion configured to contact the tissue;
a passageway defined within said first distal portion, wherein said passageway comprises a fluid-receiving portion; and
an aperture in said first distal portion, wherein said aperture is in fluid communication with said passageway; and
a second electrode comprising a second distal portion configured to contact the tissue.

20. The surgical instrument of claim 19, wherein said second distal portion of said second electrode comprises a spiral coil, and wherein said first distal portion of said first electrode is positioned within said spiral coil.
